(19) United States
(12) Patent Application Publication
(10) Pub. No.: US 2001/0005914 A1
(43) Pub. Date: Jul. 5, 2001

(54) OXIDATION DYES

(75) Inventors: ANDREAS J. BITTNER, OFFENBACH (DE); ASTRID KLEEN, ERKRATH (DE)

Correspondence Address: HENKEL CORPORATION, 2500 RENAISSANCE BLVD, STE 200, GULPH MILLS, PA, 19406, US

(73) Assignee: Andreas J. Bittner

(21) Appl. No.: 09/214,062

(22) PCT Filed: Jul. 3, 1997
(86) PCT No.: PCT/EP97/03521

§ 371 Date: None

(30) Foreign Application Priority Data
Jul. 3, 1996 (DE) 196 26 617.3
Jul. 3, 1996 (DE) 196 26 744.7
Jul. 3, 1996 (DE) 196 26 682.3

(51) Int. Cl. A61K 7/13

(52) U.S. Cl. 8/405

(57) ABSTRACT

The invention relates to oxidation colorants which are particularly suitable for coloring keratin fiber and to a method of coloring such fiber. The colorants contain as the preliminary oxidation dye at least one diamino aniline of the general formula (I), in which R₁ to R₆ independently of each other are hydrogen, a (C₁₋C₄)-alkyl group, a hydroxy-(C₂₋C₃)-alkyl group, a (C₁₋C₄)-alkoxy-(C₂₋C₃)-alkyl group, an amino-(C₂₋C₃)-alkyl group in which the amino group can also have one or two (C₁₋C₄)-alkyl radicals, or a 2,3-dihydroxypropyl group provided that not all substituents R₁ to R₆ are simultaneously hydrogen, and R₁ and R₂ and/or R₃ and R₄ and/or R₅ and R₆ along with the nitrogen atom to which they are attached are also an aziridine ring, an azetidine ring, a pyrrolidine ring, a piperidine ring, an azepane ring, an azocine ring or a morpholino group, thiomorpholino group or piperazino group which has another substituent R₇ on the nitrogen atom which is selected from hydrogen, a (C₁₋C₄)-alkyl group, a hydroxy-(C₂₋C₃)-alkyl group, a (C₁₋C₄)-alkoxy-(C₂₋C₃)-alkyl group, an amino-(C₂₋C₃)-alkyl group, or a 2,3-dihydroxypropyl group, and the three remaining hydrogen atoms on the benzol ring can also be replaced independently of each other by a halogen atom or a (C₁₋C₄)-alkyl group, or the physiologically tolerable salts thereof with inorganic and organic acids. Shades of color are obtained which have a high level of brilliancy and color fastness.

This invention relates to oxidation colorants containing special diaminoanilines as oxidation dye precursors.

By virtue of their intensive colors and good fastness properties, so-called oxidation colorants play a prominent role in the coloring of keratin fibers, particularly human hair. Oxidation colorants normally contain oxidation dye precursors, so-called primary intermediates and secondary intermediates. The primary intermediates form the actual dyes with one another or by coupling with one or more secondary intermediates in the presence of oxidizing agents or atmospheric oxygen.

Good oxidation dyes (precursors) are expected to satisfy above all the following requirements: they must form the required color tones with sufficient intensity and fastness during the oxidative coupling reaction. In addition, they must be readily absorbed onto the fibers with no significant differences—particularly in the case of human hair—between damaged and freshly regrown hair (levelling behavior). They must be resistant to light, heat and the effect of chemical reducing agents, for example permanent wave lotions. Finally, if they are used to color hair, they should not overly stain the scalp and, above all, should be toxicologically and dermatologically safe.

The primary intermediates used are, for example, primary aromatic amines containing another free or substituted hydroxy or amino group in the para position or the ortho position, diaminopyridine derivatives, heterocyclic hydrazones, 4-aminopyrazolone derivatives and 2,4,5,6-tetraaminopyrimidine and derivatives thereof.

The secondary intermediates are generally m-phenylenediamine derivatives, naphthols, resorcinol and resorcinol derivatives, pyrazolones, m-aminophenols and pyridine derivatives. With regard to the individual dye components suitable for use in accordance with the invention, reference is specifically made to the Colipa List published by the Industrieverband Korperpflege und Waschmittel, Frankfurt.

In general, natural color tones cannot be obtained with a single secondary intermediate/primary intermediate combination. In practice, therefore, a combination of various primary intermediates and secondary intermediates has to be used to obtain a natural-looking color.

Thus, many intensive blue color tones obtainable with the known primary intermediate/secondary intermediate combinations contain a distinct red component. This red component is a disadvantage, particularly in the case of lighter shades, but also for obtaining natural shades which are intended to have an adequate depth of color and an adequate grey-covering effect.

Accordingly, there is still a need for primary intermediate/secondary intermediate combinations which produce an intensive color in the clear blue range and, more particularly, a pure black tone with no tinges of blue or red.

In addition, the risk of an uneven coloring result, poorer levelling behavior and less favorable fastness properties also increases with increasing number of the oxidation dye precursors used.

Accordingly, there is still a need for new oxidation dye precursors which, in particular, even enable natural colors to be obtained using a smaller number of dye precursors.

Accordingly, the problem addressed by the present invention was to provide new compounds which would satisfy the requirements oxidation dye precursors are expected to meet to a particular degree.

It has now surprisingly been found that, by virtue of their particular electronic structure, the compounds of general formula (I) described in the present invention satisfy these requirements particularly well. In particular, “pure black” colors and very natural blond and, in particular, brown tones can be obtained with them.

In addition, these compounds surprisingly show both pronounced secondary intermediate properties and pronounced primary intermediate properties. As a result, a large number of color tones can be obtained with a small number of other oxidation dye precursors of the secondary intermediate and/or primary intermediate type without the levelling and fastness problems often observed where relatively large numbers of oxidation dye precursors are used occurring.

In a first embodiment, therefore, the present invention relates to oxidation colorants for coloring keratin fibers which contain as oxidation dye precursor at least one diaminoaniline corresponding to general formula (I):

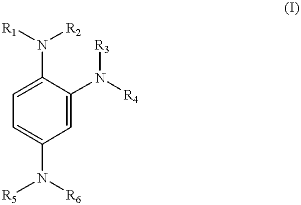

in which R₁ to R₆ independently of one another represent

hydrogen,

a (C₁₋₄)-alkyl group,

a hydroxy-(C₂₋₃)-alkyl group,

a (C₁₋₄)-alkoxy-(C₂₋₃)-alkyl group,

an amino-(C₂₋₃)-alkyl group, where the amino group may also bear one or two (C₁₋₄)-alkyl radicals, or

a 2,3-dihydroxypropyl group,

with the proviso that not all the substituents R₁ to R₆ simultaneously stand for hydrogen, and

R₁ and R₂ and/or R₃ and R₄ and/or R₅ and R₆ together with the nitrogen atom to which they are attached may also stand for an aziridine, acetidine, pyrrolidine, piperidine, azepan, azocine ring or a morpholino, thiomorpholino or piperazino group which, at the nitrogen atom, bears another substituent R₇ selected from hydrogen, a (C₁₋₄)-alkyl, a hydroxy-(C₂₋₃)-alkyl, a (C₁₋₄)-alkoxy-(C₂₋₃)-alkyl, an amino-(C₂₋₃)-alkyl or a 2,3-dihydroxypropyl group and the three remaining hydrogen atoms at the benzene ring independently of one another may even be replaced by a halogen atom or by a (C₁₋₄)-alkyl group,

or physiologically compatible salts thereof with inorganic and organic acids.

These compounds may be prepared by known methods. Specific reference is made in this regard to the Examples of the present specification.

Colorants containing a compound of formula (I) where at least two of the groups R₁ to R₆ are not hydrogen show particularly outstanding coloring properties.

Other preferred compounds of formula (I) are those in which at least one of the groups —NR₁R₂, —NR₃R₄ or —NR₅R₆ stands for an aziridine, acetidine, pyrrolidine, piperidine, azepan, azocine ring or for a morpholino, thiomorpholino or piperazino group which, at the nitrogen atom, bears another substituent R₇ selected from hydrogen, a (C₁₋₄)-alkyl, a hydroxy-(C₂₋₃)-alkyl, a (C₁₋₄)-alkoxy-(C₂₋₃)-alkyl, an amino-(C₂₋₃)-alkyl or a 2,3-dihydroxypropyl group.

Preferred groups R₁ to R₆ are hydrogen, methyl, ethyl, 2-hydroxyethyl and 3-hydroxypropyl.

Preferred groups —NR₁R₂, —NR₃R₄ and —NR₅R₆ are pyrrolidine, piperidine, azepan, morpholine and piperazine, the latter carrying hydrogen at the other nitrogen atom.

The compounds corresponding to formula (I) may be present both as free bases and in the form of their physiologically compatible salts with inorganic or organic acids, for example hydrochlorides, sulfates and hydrobromides. Other acids suitable for salt formation are phosphoric acid and also acetic acid, propionic acid, lactic acid and citric acid. Accordingly, the following observations on the compounds corresponding to formula (I) always apply to these salts also.

Keratin fibers in the context of the invention are pelts, wool, feathers and, in particular, human hair. Although the oxidation colorants according to the invention are primarily suitable for coloring kerating fibers, there is nothing in principle to stop them being used in other fields, particularly in color photography.

The hair colorants according to the invention contain the compounds corresponding to formula (I) in a quantity of preferably 0.001 to 10% by weight and, more preferably, 0.1 to 5% by weight, based on the oxidation colorant as a whole. Both here and in the following, the expressions “oxidation colorant as a whole” or “colorant as a whole” refer to the product which is presented to the user. Depending upon the particular formulation, this product may be applied to the hair either directly or after mixing with water or, for example, an aqueous solution of an oxidizing agent.

The compounds corresponding to formula (I) may act both as primary intermediates and as secondary intermediates in the oxidation colorants according to the invention.

In a first embodiment, the colorants according to the invention only contain the compounds of formula (I) as oxidation dye precursors.

However, the number of shades obtainable is distinctly increased if, in addition to the compounds of formula (I), the colorant also contains at least one other oxidation dye precursor.

In a second preferred embodiment, therefore, the colorants according to the invention additionally contain at least one other oxidation dye precursor of the secondary intermediate type.

According to the invention, preferred secondary intermediates are 1-naphthol, pyrogallol, 1,5-, 2,7- and 1,7-dihydroxynaphthalene, o-amino-phenol, 5-amino-2-methylphenol, m-aminophenol, resorcinol, resorcinol monomethyl ether, m-phenylene diamine, 1-phenyl-3-methyl-5-pyrazolone, 2,4-dichloro-3-aminophenol, 1,3-bis-(2,4-diaminophenoxy)-propane, 4-chlororesorcinol, 2-chloro-6-methyl-3-aminophenol, 2-methyl resorcinol, 5-methyl resorcinol, 2,5-dimethyl resorcinol, 2,6-dihydroxypyridine, 2,6-diaminopyridine, 2-amino-3-hydroxypyridine, 2,6-dihydroxy-3,4-diaminopyridine, 3-amino-2-methylamino-6-methoxypyridine, 4-amino-2-hydroxytoluene, 2,6-bis-(2-hydroxyethylamino)-toluene, 2,4-diaminophenoxyethanol, 2-amino-4-hydroxyethylaminoanisole.

According to the invention, 1,7-dihydroxynaphthalene, m-aminophenol, 2-methyl resorcinol, 4-amino-2-hydroxytoluene, 2-amino-4-hydroxyethyl-aminoanisole and 2,4-diaminophenoxyethanol are particularly preferred.

This embodiment does of course also encompass the use of several additional secondary intermediates. According to the invention, preferred secondary intermediate combinations are

resorcinol, m-phenylene diamine, 4-chlororesorcinol, 2-amino4-hydroxy-ethylaminoanisole,

2-methyl resorcinol, 4-chlororesorcinol, 2-amino-3-hydroxypyridine,

resorcinol, m-aminoaniline, 2-hydroxy-4-aminotoluene,

3-methyl-4-aminoaniline, m-aminoaniline, 2-hydroxy4-aminotoluene, 2-amino-3-hydroxypyridine,

2-methyl resorcinol, m-aminoaniline, 2-hydroxy4-aminotoluene, 2-amino-3-hydroxypyridine.

In a second preferred embodiment, therefore, the colorants according to the invention optionally contain at least one other oxidation dye precursor of the primary intermediate type in addition one other oxidation dye precursor of the secondary intermediate type.

According to the invention, preferred primary intermediates are p-phenylene diamine, p-toluylene diamine, p-aminophenol, 3-methyl-1,4-diaminobenzene, 1-(2′-hydroxyethyl)-2,5-diaminobenzene, N,N-bis-(2-hydroxyethyl)-p-phenylene diamine, 2-(2,5-diaminophenoxy)-ethanol, 1-phenyl-3-carboxyamido-4-amino-5-pyrazolone, 4-amino-3-methyl phenol, 2-methylamino4-aminophenol, 2,4,5,6-tetraaminopyrimidine, 2-hydroxy-4,5,6-triaminopyrimidine, 4-hydroxy-2,5,6-triaminopyrimidine, 2,4-dihydroxy-5,6-diaminopyrimidine, 2-dimethylamino-4,5,6-triaminopyrimidine and 2-hydroxyethylaminomethyl-4-aminophenol.

According to the invention, p-toluylene diamine, p-aminophenol, 1-(2′-hydroxyethyl)-2,5-diaminobenzene, 4-amino-3-methylphenol, 2-methylamino-4-aminophenol and 2,4,5,6-tetraaminopyrimidine are most particularly preferred.

This embodiment does of course also encompass the use of several additional primary intermediates. According to the invention, preferred primary intermediate combinations are

p-toluylene diamine, p-phenylene diamine,

3-methyl-4-aminoaniline, p-toluylene diamine,

p-toluylene diamine, 4-amino-3-methylphenol,

p-toluylene diamine, 2-methylamino-4-aminophenol,

2,4, 5,6-tetraaminopyrimidine, 1 -(2′-hydroxyethyl)-2, 5-diaminobenzene,

2,4,5,6-tetraaminopyrimidine, p-toluylene diamine.

The primary and secondary intermediates are normally used in a substantially equimolar ratio to one another. Although it has proved to be useful to employ the primary and secondary intermediates in an equimolar ratio, a certain excess of individual oxidation dye precursors is by no means a disadvantage, so that the primary and secondary intermediates may advantageously be present in the colorant in a molar ratio of 1:0.5 to 1:2. The total quantity of oxidation dye precursors is generally at most 20% by weight, based on the colorant as a whole.

In a fourth, likewise preferred embodiment, the colorants according to the invention optionally contain substantive dyes in addition to other oxidation dye precursors for further modifying the color tones. The substantive dyes in question belong, for example, to the group consisting of nitrophenylene-diamines, nitroaminophenols, anthraquinones or indophenols. Preferred substantive dyes are the compounds known under the International names or commercial names of HC Yellow 2, HC Yellow 4, Basic Yellow 57, Disperse Orange 3, HC Red 3, HC Red BN, Basic Red 76, HC Blue 2, Disperse Blue 3, Basic Blue 99, HC Violet 1, Disperse Violet 1, Disperse Violet 4, Disperse Black 9, Basic Brown 16, Basic Brown 17, picramic acid and Rodol R and also 4-amino-2-nitrodiphenylamine-2′-carboxylic acid, 6-nitro-1,2,3,4-tetrahydroquinoxaline, (N-2,3-dihydroxypropyl-2-nitro-4-trifluoromethyl)-aminobenzene and 4-N-ethyl-1,4-bis-(2′-hydroxyethylamino)-2-nitrobenzene hydrochloride. The colorants according to this embodiment of the invention contain the substantive dye in a quantity of preferably 0.01 to 20% by weight, based on the colorant as a whole.

In addition, the colorants according to the invention may also contain naturally occurring dyes such as, for example, henna red, henna neutral, henna black, camomile blossom, sandalwood, black tea, black alder bark, sage, logwood, madder root, catechu, sedre and alkanet.

The oxidation dye precursors compulsorily or optionally present do not have to be single compounds. Instead, the hair colorants according to the invention—due to the processes used for producing the individual dyes—may contain small quantities of other components providing they do not adversely affect the coloring result or have to be ruled out for other reasons, for example toxicological reasons.

Typical formulations for the oxidation colorants according to the invention are preparations based on water or non-aqueous solvents and powders.

In one preferred embodiment for the production of the colorants according to the invention, the oxidation dye precursors are incorporated in a suitable water-containing carrier. For coloring hair, such carriers are, for example, cremes, emulsions, gels or even surfactant-containing foaming solutions, for example shampoos, foam aerosols or other formulations suitable for application to the hair. The hair colorants according to the invention are adjusted to a pH value of preferably 6.5 to 11.5 and, more preferably, 9 to 10.

The colorants according to the invention may also contain any of the known active substances, additives and auxiliaries typical of such formulations. In many cases, the colorants contain at least one surfactant, both anionic and zwitterionic, ampholytic, nonionic and cationic surfactants being suitable in principle. In many cases, however, it has been found to be of advantage to select the surfactants from anionic, zwitterionic or nonionic surfactants. Anionic surfactants can be particularly useful.

Suitable anionic surfactants for the hair colorants according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ether, amide and hydroxyl groups and—generally—ester groups may also be present in the molecule. The following are examples of suitable anionic surfactants—in the form of the sodium, potassium and ammonium salts and the mono-, di- and trialkanolammonium salts containing 2 or 3 carbon atoms in the alkanol group:

linear and branched fatty acids containing 8 to 22 carbon atoms (soaps),

ether carboxylic acids corresponding to the formula R—O—(CH₂-CH₂O)ₓ—CH₂—COOH, in which R is a linear alkyl group containing 10 to 22 carbon atoms and x=0 or 1 to 16,

acyl sarcosides containing 10 to 18 carbon atoms in the acyl group,

acyl taurides containing 10 to 18 carbon atoms in the acyl group,

acyl isethionates containing 10 to 18 carbon atoms in the acyl group,

sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,

linear alkane sulfonates containing 12 to 18 carbon atoms,

linear α-olefin sulfonates containing 12 to 18 carbon atoms,

α-sulfofatty acid methyl esters of fatty acids containing 12 to 18 carbon atoms,

alkyl sulfates and alkyl polyglycol ether sulfates corresponding to the formula R—O(CH₂—CH₂O)ₓ—SO₃H, in which R is a preferably linear alkyl group containing 10 to 18 carbon atoms and x=0 or 1 to 12,

mixtures of surface-active hydroxysulfonates according to DE-A-37 25 030,

sulfated hydroxyalkyl polyethylene and/or hydroxyalkylene propylene glycol ethers according to DE-A-37 23 354,

sulfonates of unsaturated fatty acids containing 12 to 24 carbon atoms and 1 to 6 double bonds according to DE-A-39 26 344,

esters of tartaric acid and citric acid with alcohols in the form of addition products of around 2 to 15 molecules of ethylene oxide and/or propylene oxide with fatty alcohols containing 8 to 22 carbon atoms.

Preferred anionic surfactants are alkyl sulfates, alkyl polyglycol ether sulfates and ether carboxylic acids containing 10 to 18 carbon atoms in the alkyl group and up to 12 glycol ether groups in the molecule and, in particular, salts of saturated and, more particularly, unsaturated C₈₋₂₂ carboxylic acids, such as oleic acid, stearic acid, isostearic acid and palmitic acid.

In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N, N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the CTFA name of Cocamidopropyl Betaine.

Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl aminopropionate, cocoacyl aminoethyl aminopropionate and C₁₂₋₁₈ acyl sarcosine.

Nonionic surfactants contain, for example, a polyol group, a poly-alkylene glycol ether group or a combination of polyol and polyglycol ether groups as the hydrophilic group. Examples of such compounds are

products of the addition of 2 to 30 moles of ethylene oxide and/or 0 to 5 moles of propylene oxide to linear fatty alcohols containing 8 to 22 carbon atoms, to fatty acids containing 12 to 22 carbon atoms and to alkylphenols containing 8 to 15 carbon atoms in the alkyl group,

C₁₂₋₂₂ fatty acid monoesters and diesters of products of the addition of 1 to 30 moles of ethylene oxide to glycerol,

C₈₋₂₂ alkyl mono- and oligoglycosides and ethoxylated analogs thereof,

products of the addition of 5 to 60 moles of ethylene oxide to castor oil and hydrogenated castor oil,

products of the addition of ethylene oxide to sorbitan fatty acid esters,

products of the addition of ethylene oxide to fatty acid alkanolamides.

Examples of cationic surfactants suitable for use in the hair treatment formulations according to the invention are, in particular, quaternary ammonium compounds. Preferred quaternary ammonium compounds are ammonium halides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride. Other cationic surfactants suitable for use in accordance with the invention are the quaternized protein hydrolyzates.

Also suitable for use in accordance with the invention are cationic silicone oils such as, for example, the commercially available products Q2-7224 (manufacturer: Dow Corning; a stabilized trimethyl silyl amodimethi-cone), Dow Corning® 929 Emulsion (containing a hydroxylamino-modified silicone which is also known as Amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80).

Alkyl amidoamines, particularly fatty acid amidoamines, such as the stearyl amidopropyl dimethyl amine obtainable as Tego Amid®S 18, are distinguished not only by their favorable conditioning effect, but also and in particular by their ready biodegradability.

Quaternary ester compounds, so-called “esterquats”, such as the dialkyl ammonium methosulfates and methyl hydroxyalkyl dialkoyloxyalkyl ammonium methosulfates marketed under the trade name of Stepantex® and the corresponding products of the Dehyquart® series, are also readily biodegradable.

One example of a quaternary sugar derivative suitable for use as a cationic surfactant is the commercially available product Glucquat®100 (CTFA name: Lauryl Methyl Gluceth-10 Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

Other active substances, auxiliaries and additives are, for example,

nonionic polymers such as, for example, vinyl pyrrolidone/vinyl acrylate copolymers, polyvinyl pyrrolidone and vinyl pyrrolidone/vinyl acetate copolymers and polysiloxanes,

cationic polymers, such as quaternized cellulose ethers, polysiloxanes containing quaternary groups, dimethyl diallyl ammonium chloride polymers, acrylamide/dimethyl diallyl ammonium chloride copolymers, dimethyl aminoethyl methacrylate/vinyl pyrrolidone copolymers quaternized with diethyl sulfate, vinyl pyrrolidone/imidazolinium methochloride copolymers and quaternized polyvinyl alcohol,

zwitterionic and amphoteric polymers such as, for example, acrylamido-propyl/trimethyl ammonium chloride/acrylate copolymers and octyl acrylamide/methyl methacrylate/tert.butyl aminoethyl methacrylate/2-hydroxypropyl methacrylate copolymers,

anionic polymers such as, for example, polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert.butyl acrylamide terpolymers,

thickeners, such as agar agar, guar gum, alginates, xanthan gum, gum arabic, karaya gum, carob bean flour, linseed gums, dextrans, cellulose derivatives, for example methyl cellulose, hydroxyalkyl cellulose and carboxymethyl cellulose, starch fractions and derivatives, such as amylose, amylopectin and dextrins, clays such as, for example, bentonite or fully synthetic hydrocolloids such as, for example, polyvinyl alcohol,

structurants, such as glucose, maleic acid and lactic acid,

hair-conditioning compounds, such as phospholipids, for example soya lecithin, egg lecithin and kephalins, and also silicone oils,

protein hydrolyzates, more particularly elastin, collagen, keratin, milk protein, soya protein and wheat protein hydrolyzates, condensation products thereof with fatty acids and quaternized protein hydrolyzates,

perfume oils, dimethyl isosorbide and cyclodextrins,

solubilizers, such as ethanol, isopropanol, ethylene glycol, propylene glycol, glycerol and diethylene glycol,

antidandruff agents, such as Piroctone Olamine and Zinc Omadine,

alkalizing agents such as, for example, ammonia, monoethanolamine, 2-amino-2-methylpropanol and 2-amino-2-methylpropane-1,3-diol,

other substances for adjusting the pH value,

active substances, such as panthenol, pantothenic acid, allantoin, pyrrolidone carboxylic acids and salts thereof, plant extracts and vitamins,

cholesterol,

UV absorbers,

consistency promoters, such as sugar esters, polyol esters or polyol alkyl ethers,

fats and waxes, such as spermaceti, beeswax, montan wax, paraffins, fatty alcohols and fatty acid esters,

fatty acid alkanolamides,

complexing agents, such as EDTA, NTA and phosphonic acids,

swelling and penetration agents, such as glycerol, propylene glycol monoethyl ether, carbonates, hydrogen carbonates, guanidines, ureas and primary, secondary and tertiary phosphates,

opacifiers, such as latex,

pearlescers, such as ethylene glycol mono- and distearate,

propellents, such as propane/butane mixtures, N₂O, dimethyl ether, CO₂ and air and

antioxidants.

To produce the colorants according to the invention, the constituents of the water-containing carrier are used in the usual quantities for this purpose. For example, emulsifiers are used in concentrations of 0.5 to 30% by weight while thickeners are used in concentrations of 0.1 to 25% by weight, based on the colorant as a whole.

Basically, the color is oxidatively developed with atmospheric oxygen or with an oxidizing agent present in or added to the colorant immediately before application.

In a first preferred embodiment, a chemical oxidizing agent is used. This is particularly advantageous in cases where human hair is to be not only colored, but also lightened. Particularly suitable oxidizing agents are hydrogen peroxide or addition products thereof with urea, melamine or alkali metal borate. In a particularly preferred variant of this embodiment, the colorant according to the invention is mixed immediately before application with the preparation of an oxidizing agent, more particularly an aqueous H₂O₂ solution. The ready-to-use hair coloring preparation formed should preferably have a pH value of6 to 10. In a particularly preferred embodiment, the hair colorant is used in a mildly alkaline medium. The application temperatures may be in the range from 15 to 40° C. After a contact time of about 30 minutes, the hair colorant is removed from the hair to be colored by rinsing. There is no need for the hair to be washed with a shampoo where a carrier of high surfactant content, for example a coloring shampoo, has been used.

In the particular case of hair which is difficult to color, the preparation containing the oxidation dye precursors may be applied to the hair without preliminary mixing with the oxidation component. The oxidation component is applied after a contact time of 20 to 30 minutes, optionally after rinsing. After another contact time of 10 to 20 minutes, the hair is rinsed and, if desired, shampooed.

In a second embodiment, the color is developed with atmospheric oxygen. In this case, it is of advantage to add an oxidation catalyst to the colorant according to the invention. Suitable oxidation catalysts are metal salts and metal complexes, transition metals being preferable. Copper, manganese, cobalt, selenium, molybdenum, bismuth and ruthenium compounds are preferred. Copper(II) chloride, sulfate and acetate can be preferred oxidation catalysts. Preferred metal complexes include the complexes with ammonia, ethylenediamine, phenanthroline, triphenyl phosphine, 1,2-diphenyl phosphinoethane, 1,3-diphenyl phosphinopropane or amino acids. The metal salts or metal complexes are present in the colorants according to the invention in quantities of preferably 0.0001 to 1 % by weight, based on the colorant as a whole. The same colorant may of course also contain several oxidation catalysts. Particulars of the production of suitable catalysts can be found in the corresponding disclosure of EP 0 709 365 A1 (page 4, lines 19 to 42) to which reference is expressly made.

The oxidation may also be carried out with enzymes. In this case, the enzymes may be used both to produce oxidizing per compounds and to enhance the effect of an oxidizing agent present in small quantities. One example of an enzymatic process is the procedure where the effect of small quantities (for example 1% and less, based on the colorant as a whole) of hydrogen peroxide is enhanced by peroxidases.

The present invention also relates to the use of diaminoanilines corresponding to general formula (I) in claim 1 for coloring keratin fibers.

The following Examples are intended to illustrate the invention.

### EXAMPLES

1. Production processes.

1.1. General production processes

1.1.1. General production process starting from 2,4-dinitrohalobenzenes

In a first process, the compounds according to the invention corresponding to general formula (I) are prepared by reacting 2,4-dinitrohalo-benzenes corresponding to general formula (II), where X=fluorine, chlorine, bromine or iodine, with amines corresponding to general formula (Ill), where R₁ and R₂ are as defined in claim 1, in an alkaline reaction medium, optionally in the presence of phase transfer catalysts, to form 2,4-dinitroanilines corresponding to general formula (IV). Suitable phase transfer catalysts are, for example, methyl or benzyl tri(C₆₋₈)alkyl ammonium chloride. This reaction may optionally be carried out under pressure in an autoclave if the boiling point of the amine is lower than the reaction temperature or if the reaction is otherwise incomplete. The compounds corresponding to general formula (IV) are reduced to the compounds corresponding to general formula (V), optionally alkylated or alkoxylated to the compounds of general formula (I) according to the invention and optionally converted into their salts with inorganic or organic acids.

The compounds corresponding to general formula (III) are standard chemical starting materials and are commercially obtainable.

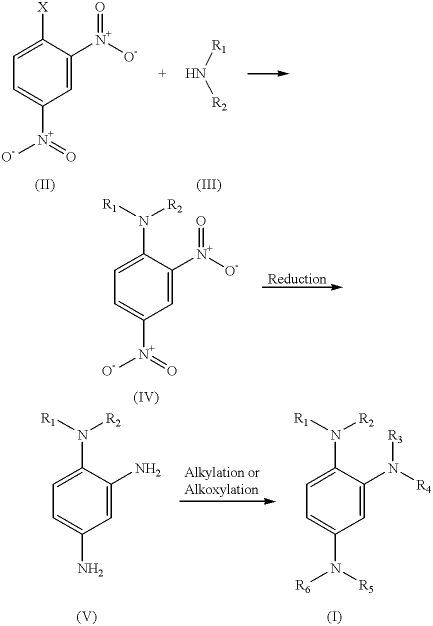

1.1.2. General production process starting from 4-amino-2-nitrohalo-benzenes

In a second process, the compounds according to the invention corresponding to general formula (I) may be obtained by initially reacting substituted 4-amino-2-nitrohalobenzenes corresponding to general formula (VI) with amines corresponding to general formula (III) to form compounds corresponding to general formula (VII):

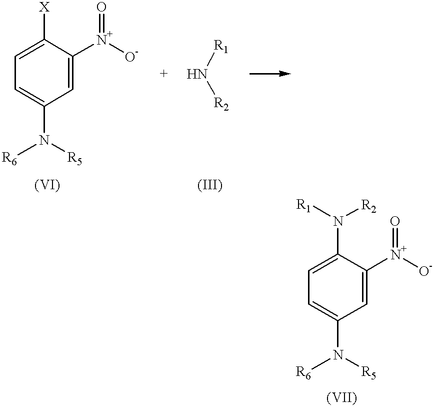

The compounds corresponding to general formula (VII) are then converted by reduction and, optionally, subsequent alkylation or alkoxylation into the compounds corresponding to general formula (I).

1.1.3. General production process starting from 2-amino-4-nitrohalo-benzenes

In a third process, the compounds corresponding to general formula (I) according to the invention may be obtained by initially reacting substituted 2-amino-4-nitrohalobenzenes corresponding to general formula (VIa) with amines corresponding to general formula (III) to form compounds correeponding to general formula (VIIa).

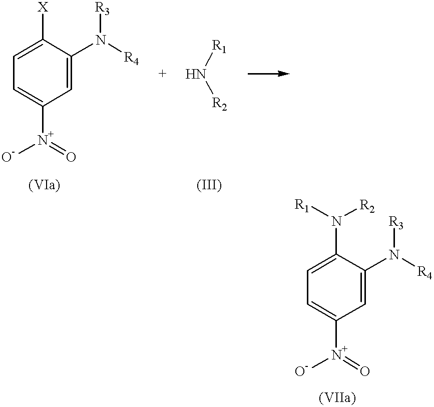

The compounds corresponding to general formula (VIIa) are converted by reduction and optionally subsequent alkylation or alkoxylation into the compounds corresponding to general formula (I).

1.1.4. General production process starting from 3-amino-4-nitrohalo-benzenes

In a fourth process, the compounds according to the invention corresponding to general formula (I) may be obtained by initially reacting substituted 3-amino4-nitrohalobenzenes corresponding to general formula (VIb) with amines corresponding to general formula (IIIb) to form compounds corresponding to general formula (VIIb):

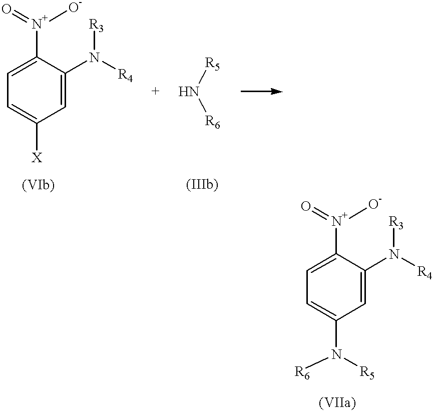

After reduction and optionally further alkylation or alkoxylation, the compounds according to the invention corresponding to general formula (I) are obtained and are optionally converted with an inorganic or organic acid into a salt.

1.1.5. General production process starting from 2-nitro-5-acetylaminohalo-benzenes

The compounds according to the invention corresponding to general formula (i) are prepared by reacting 2-nitro-5-acetylaminohalobenzenes corresponding to general formula (II)′, where X=fluorine, chlorine, bromine or iodine, with amines corresponding to general formula (IIIa), where R₃ and R₄ are as defined above, in an alkaline reaction medium, optionally in the presence of phase transfer catalysts, to form 2-nitro-5-acetylaminoanilines corresponding to general formula (IV)′. The 2-nitro-5-acetylaminoanilines (IV)′are hydrolyzed to the compounds of general formula (V)′ and optionally alkylated or alkoxylated and then further reduced and optionally alkylated or alkoxylated to the compounds according to the invention corresponding to general formula (I). Suitable phase transfer catalysts are, for example, methyl or benzyl tri(C₆₋₈)alkyl ammonium chloride. This reaction may optionally be carried out under pressure in an autoclave if the boiling point of the amine is lower than the reaction temperature or if the reaction is otherwise incomplete.

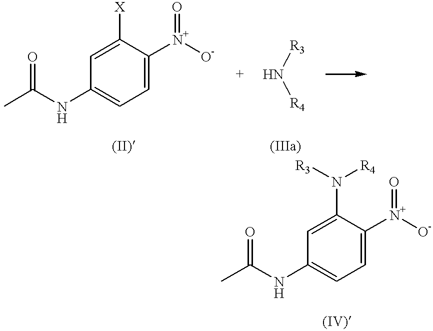

The compounds corresponding to general formula (IIIa) are typical chemical starting materials and are commercially obtainable. The compounds corresponding to general formula (IV)′ are converted into the compounds of general formula (I) by hydrolysis and optionally alkylation or alkoxylation, reduction and optionally further alkylation or alkoxylation and are optionally converted with acids into their salts.

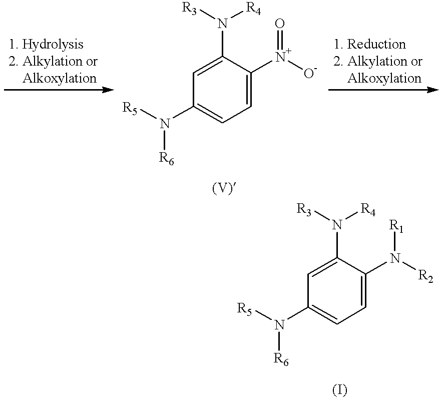

1.1.6. General production process starting from 2-nitro-5-aminohalo-benzenes

In another process, the compounds according to the invention corresponding to general formula (I) may be obtained by initially reacting substituted 2-nitro-5-aminohalobenzenes corresponding to general formula (IV)′, where R₅ and R₆ are as defined in claim 1, with amines corresponding to general formula (IIIa) to form compounds corresponding to general formula (VIIb):

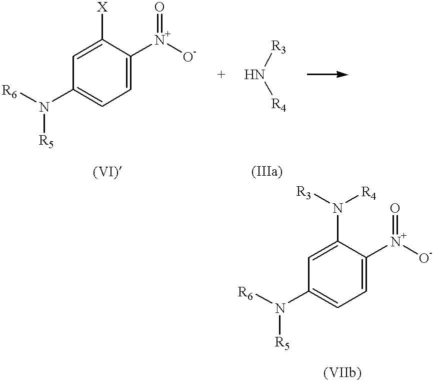

The compounds corresponding to general formula (VIIb) are then converted into the compounds corresponding to general formula (I) by reduction and optionally subsequent alkylation or alkoxylation.

1.1.7. General production process starting from 4-nitro-3-acetaminohalo-benzenes

The compounds according to the invention corresponding to general formula (I) are prepared by reacting 4-nitro-3-acetaminohalobenzenes corresponding to general formula (II)″, where X=fluorine, chlorine, bromine or iodine, with amines corresponding to general formula (IIIb), where R₅ and R₆ are as defined above, in an alkaline reaction medium, optionally in the presence of phase transfer catalysts, to form substituted 4-nitro-3-acetamino-anilines corresponding to general formula (IV)″. Suitable phase transfer catalysts are, for example methyl or benzyl tri(C₆₋₈)alkyl ammonium chloride. This reaction may optionally be carried out under pressure in an autoclave if the boiling point of the amine is lower than the reaction temperature or if the reaction is otherwise incomplete. The compounds corresponding to general formula (IV)″ are hydrolyzed to the compounds corresponding to general formula (VIIb) and, optionally after alkylation or alkoxylation, are reduced and further alkylated or alkoxylated to the compounds according to the invention corresponding to general formula (I) and are optionally converted with inorganic or organic acids into their salts. The compounds corresponding to general formula (IIIb) are standard chemical starting materials and are commercially obtainable.

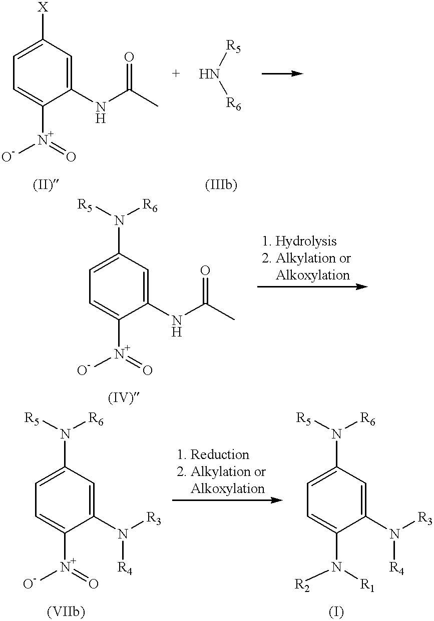

1.2. General observations on the production processes

The first stage of these processes essentially comprises exchanging a halogen substituent for an amine substituent at the phenyl ring. The known processes are normally carried out with an excess of amine of about 40 to 80%. The products are obtained in yields of about 90% and with a purity of 95 to 96%. It has now surprisingly been found that higher yields can be obtained for the same or better purities and a faster conversion if the excess of amine is 30% or less, more particularly 5 to 10 mole-%, based on the quantities of compound (II), (VI), (VIa), (VIb), (II)′, (VI)′ and (II)″ used. The reaction of the amines (III), (IIIa) or (IIIb) with the compounds (II), (VI), (VIa), (VIb), (II)′ (VI)′ and (II)″ is preferably carried out in the presence of alkali metal carbonates as acid-binding agents. In another preferred embodiment, the reaction is carried out in an organic solvent. The reaction is also preferably carried out in the presence or one or more phase transfer catalysts, for example methyl or benzyl tri(C₆₋₈)alkyl ammonium chloride. Finally, the reaction is preferably carried out under a pressure of 1 to 15 bar, more preferably under a pressure of 1 to 8 bar and most preferably under a pressure of 1 to 2.5 bar.

The compounds corresponding, for example, to general formulae (VI), (VIa), (VIb), (V)′ and (VIIb) can be obtained by alkylation or alkoxylation of compounds corresponding to formulae (VI), (VIa), (VIb), (V)′ and (VIIb), where R₃ and R₄ or R₅ and R₆=hydrogen. This can be done by reacting these compounds with dialkyl sulfate, alkyl halide or alkylene oxides in an inert solvent or by rearranging carbamates obtained therefrom and subsequently treating them with the alkylating agents mentioned above.

The reaction of, for example, compounds corresponding to formula (V)′, (VII), (VIIa) or (VIIb) [R₃ and R₄=hydrogen or R₅ and R₆=hydrogen] with chloroformic acid-2-chloroethyl ester or chloroformic acid-3-chloropropyl ester may be carried out on the lines of the known selective hydroxyalkylation of an amine with chloroformic acid chloroalkyl ester and subsequent treatment of the chloroalkyl carbamates with a base. In this process, for example, a compound corresponding to formula (VII), (VIIa) or (VIIb), where R₃, R₄, R₅ and R₆ represent hydrogen,

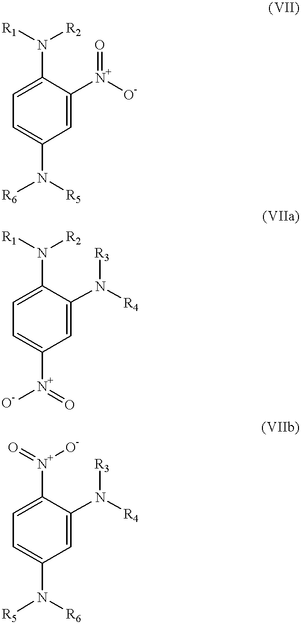

is reacted in an inert solvent with chloroformic acid-2-chloroethyl ester or chloroformic acid-3-chloropropyl ester to form compounds corresponding to general formula (VIII), (VIIIa) or (VIIIb), where R₇=CH₂CH₂Cl or CH₂CH₂₋CH₂Cl:

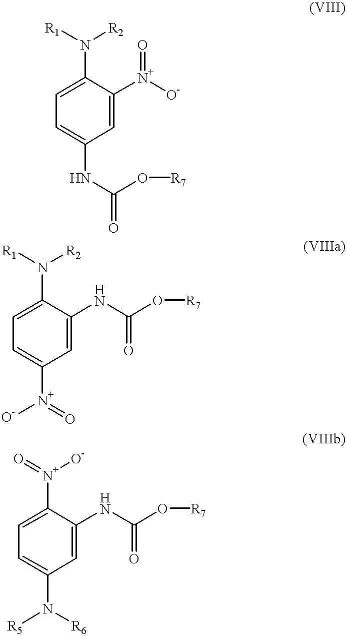

which are reacted in a solvent with bases to form compounds corresponding to general formula (IX), (IXa) or (IXb), where R₈=CH₂CH₂OH or CH₂CH₂₋CH₂OH:

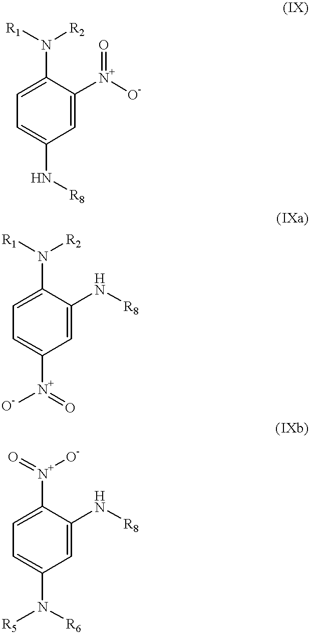

which are reacted with known alkylating or alkoxylating agents to form compounds corresponding to general formula (VII), (VIIa) or (VIIb), where R₁ to R₆ are as already defined, which—after reduction and optionally after further alkylation or alkoxylation—give the compounds corresponding to general formula (I).

The compounds corresponding to general formula (I) may be produced by reducing the compounds corresponding to general formula (V)′, (IV), (VII), (VIIa) or (VIIb), with base metals or by catalytic reduction, optionally after alkylation or alkoxylation.

The catalytic reduction is carried out with standard catalysts, for example Raney nickel, palladium on active carbon or platinum on active carbon. The reaction temperature is between room temperature and 120° C. and preferably between 35 and 100° C. while the pressure is between normal pressure and 20 bar and preferably between 2 and 7 bar. The solvents used are standard solvents, such as water, toluene, glacial acetic acid, lower alcohols or ethers. After the reduction and removal of the catalyst, the product corresponding to general formula (I), may be isolated in free form by distilling off the solvent in an inert gas atmosphere, optionally after alkylation or alkoxylation. Suitable alkylating agents are the known compounds dimethyl and diethyl sulfate while suitable alkoxylating agents are the known compounds ethylene oxide and propylene oxide. The product corresponding to general formula (I) is converted into a salt, preferably in an inert gas atmosphere, by adding a 1.0- to 1.1-equivalent quantity of an acid. The salt either precipitates directly or is obtained after removal of the solvent.

Suitable inorganic acids for salt formation are, for example, hydrochloric acid, sulfuric acid, phosphoric acid while suitable organic acids for salt formation are acetic acid, propionic acid, lactic acid or citric acid.

1.3. Preparation of special compounds corresponding to formula (I)

The compounds prepared were characterized by IR spectra or IR(KBr pellet) and ¹H-NMR spectra (in D₆-DMSO). In the case of the IR spectra, only the very strong and strong bands are mentioned. In the data on the ¹H-NMR spectra, s=singlet, d=doublet, dd=doublet of the doublet, t=triplet, q=quartet, qi—quintet, m=multiplet, ³J and ⁴J=the couplings via three or four bonds and H², H³, H⁴, H⁵ and H⁶=the hydrogen atoms in positions 2, 3, 4, 5 and 6 of the benzene ring.

1.3.1. Preparation of N,N-dimethyl-2,4-diaminoaniline sulfate

Step a) N,N-dimethyl-2,4-dinitroaniline

97.3 g (0.5 mole) of 2,4-dinitroaniline were dissolved in 500 ml of dimethyl sulfoxide and 141.9 g (1.0 mole) of methyl iodide were added dropwise with stirring to the resulting solution. The mixture was stirred until everything had dissolved, after which 112.2 g (1.0 mole) of 50% potassium hydroxide were slowly added dropwise. The mixture was then left to cool while stirring to room temperature and then in an ice bath to 10° C., the product precipitating. The product precipitated was filtered off under suction, washed twice with about 100 ml of water and dried in vacuo at 40° C.

| | |
|---|---|
| Yield: | 92.1 g(87.2% of the theoretical) |
| Melting point: | 90° C. (decomp.) |
| IR: | 3356 cm⁻¹(v CH_{Ar}), 3115, 2928 cm⁻¹(v CH), 1622 |
| | cm⁻¹(v C═C), 1587, 1523 cm⁻¹(vₐₛ NO₂), 1337, 1311 |
| | cm⁻¹(vₛ NO₂). |
| ¹H-NMR: | 8.83 ppm(H³, d, ⁴J_{H,H}=2.68 Hz); 8.27 ppm(H⁵, dd, |
| | ³J_{H,H}=9.58 Hz, ⁴J_{H,H}=2.59 Hz); 7.27 ppm(H⁶, d, |
| | ³J_{H,H}=9.59 Hz); 3.07 ppm(3H, s, syn-NC+E,uns H₃) 3.05 |
| | ppm(3H, s, anti-NC+E,uns H₃). |

Step b) N,N-dimethyl-Z4-diaminoaniline sulfate

150 ml of methanol were introduced into a 0.3 liter autoclave, 42.2 g (0.2 mole) of N,N-dimethyl-2,4-dinitroaniline (step a; alternatively even the compound of Example 1.3.8 step a) were dissolved therein and 2 g of palladium on active carbon 10% (Degussa) were added. After the autoclave had been closed and blanketed with nitrogen, hydrogenation was carried out under a pressure of 3 bar and at a temperature of 35 to 40° C. until no more hydrogen was taken up. 1.3 g of active carbon were added under nitrogen to the warm solution and the catalyst was filtered off. 37 g of 80% sulfuric acid (alternatively 32 ml of concentrated hydrochloric acid per 0.2 mole) were added dropwise to the solution at 5° C. while cooling with ice. The product precipitated was filtered under suction, washed with methanol and dried.

| | |
|---|---|
| Yield | 39.9 g(80% of the theoretical) |
| Melting point: | >250° C. |
| IR: | 3397 cm⁻¹(v OH), 3234 cm⁻¹(v CH_{Ar}), 2920 cm⁻¹(v |
| | CH_{Alkyl}), 1661, 1630, 1515 cm⁻¹(v NH₃⁺), 1596 cm⁻¹(v |
| | C═C). |
| ¹H-NMR | 7.65-4.75 ppm(6H, NH₃⁺); 6.65 ppm(H⁶, d, ³J_{H,H}=8.34 |
| | Hz); 6.51 ppm(H⁵, dd, ³J_{H,H}=8.48 Hz, ⁴J_{H,H}=2.34 Hz); |
| | 6.46 ppm(H³, d, ⁴J_{H,H}=2.29 Hz); 2.77 ppm(6H, s, |
| | N(CH₃)₂). |

1.3.2. Preparation of N,N-diethyl-2,4-diaminoaniline trihydrochloride

Step a) N,N-diethyl-2,4-dinitroaniline

20.26 g (0.1 mole) of 2,4-dinitrochlorobenzene were dissolved in 150 ml of dimethyl sulfoxide^{[*]}, 8.3 g (0.06 mole) of potassium carbonate were added and 10.5 g (0.14 mole) of diethyl amine were added dropwise with stirring. The mixture was stirred at 80° C. until the reaction was complete. The mixture was poured onto 800 ml of an ice/water mixture and stirred, the product precipitating. The product precipitated was filtered under suction, washed twice with about 100 ml of water and then dried in vacuo at 40° C.

^{[*]}This reaction may also be carried out with advantage in 1,2-dimethoxy-ethane.

| | |
|---|---|
| Yield: | 21.9 g(90.8% of the theoretical) |
| Melting point: | 75-77° C. |
| IR: | 3117 cm⁻¹(v CH_{Ar}), 2983, 2930 cm⁻¹(v CH), 1607 |
| | cm⁻¹(v C═C), 1576, 1528 cm⁻¹(vₐₛ NO₂), 1321 cm⁻¹(vₛ |
| | NO₂). |
| ¹H-NMR: | 8.55 ppm(H³, d, ⁴J_{H,H}=2.80 Hz); 8.22 ppm(H⁵, dd, |
| | ³J_{H,H}=9.58 Hz, ⁴J_{H,H}=2.81 Hz); 7.36 ppm(H⁶, d, |
| | ³J_{H,H}=9.58 Hz); 3.35 ppm(4H, q, NC+E,uns H₂) 1.16 ppm(6H, t, |
| | NCH₂C+E,uns H₃). |

Step b) N,N-diethyl-2,4-diaminoaniline trihydrochloride

The reaction of the product obtained in step a) was carried out in the same way as Example 1.3.1. step b) by catalytic reduction and subsequent precipitation with hydrochloric acid.

| | |
|---|---|
| Yield: | 8 g(34.6% of the theoretical). |

1.3.3. Preparation of N-(2,4-diaminophenyl)morpholine sulfate

Step a) N-(2,4-dinitrophenyl)morpholine

Step a) was carried out in the same way as Example 1.3.1. step a) by reacting 2,4-dinitrochlorobenzene with morpholine.

| | |
|---|---|
| Yield: | 24.0 g(94.0% of the theoretical) |
| Melting point: | 104-105° C. |
| IR: | 3093 cm⁻¹(v CH_{Ar}), 2988, 2919, 2865 cm⁻¹(v CH), 1606 |
| | cm⁻¹(v C═C), 1532, 1504 cm⁻¹(vₐₛ NO₂), 1327 cm⁻¹(vₛ |
| | NO₂). |
| ¹H-NMR: | 8.63 ppm(H³, d, ⁴J_{H,H}=2.74 Hz); 8.31 ppm(H⁵, dd, |
| | ³J_{H,H}=9.37 Hz, ⁴J_{H,H}=2.75 Hz); 7.45 ppm(H⁶, d, |
| | ³J_{H,H}=9.46 Hz); 3.74 ppm(4H, t, ³J_{H,H}=4.68 Hz, OCH₂) |
| | 3.31 ppm(4H, q, NCH₂). |

Step b) N-(2,4-diaminophenyl)morpholine sulfate

Step b) was carried out in the same way as Example 1.3.1. step b) by catalytic reduction of the product obtained in step a) and precipitation with sulfuric acid.

| | |
|---|---|
| Yield: | 18.0 g(74.3% of the theoretical) |
| Melting point: | 176-178° C. |
| IR: | 3351 cm⁻¹(v CH_{Ar}), 2860, 2567 cm⁻¹(v CH), 1560 |
| | cm⁻¹(v C═C). |
| ¹H-NMR: | 8.35 ppm(H⁶, d, ³J_{H,H}=8.54 Hz); 7.22 ppm(H³, d, |
| | ³J_{H,H}=2.29 Hz); 7.08 ppm(H⁵, dd, ³J_{H,H}=8.47 Hz, |
| | ⁴J_{H,H}=2.31 Hz); 3.81 ppm(4H, t, ³J_{H,H}=4.29 Hz NCH₂); |
| | 2.93 ppm(4H, t, ³J_{H,H}=4.21 Hz, NCH₂C+E,uns H₂). |

1.3.4. Preparation of N-(2,4-diaminophenyl)piperidine sulfate

Step a) N-(2,4-dinitrophenyl)piperidine

Step a) was carried out in the same way as Example 1.3.1. step a) by reacting 2,4-dinitrochlorobenzene with piperidine.

| | |
|---|---|
| Yield: | 24.8 g(98.0% of the theoretical) |
| Melting point: | 88-91° C. |
| IR: | 3110 cm⁻¹(v CH_{Ar}), 2949, 2927, 2861 cm⁻¹(v CH), 1604 |
| | cm⁻¹(v C═C), 1525, 1505 cm⁻¹(vₐₛ NO₂), 1325 cm⁻¹(vₛ |
| | NO₂). |
| ¹H-NMR: | 8.60 ppm(H³, d, ⁴J_{H,H}=2.82 Hz); 8.25 ppm(H⁵, dd, |
| | ³J_{H,H}=9.42 Hz, ⁴J_{H,H}=2.76 Hz); 7.41 ppm(H⁶, d, |
| | ³J_{H,H}=9.49 Hz); 3.27 ppm(4H, s, NC+E,uns H₂); 1.65 ppm(6H, |
| | m, NCH₂C+E,uns H₂C+E,uns H₂C+E,uns H₂) |

Step b) N-(2,4-diaminophenyl)piperidine sulfate

Step b) was carried out in the same way as Example 1.3.1. step b) by catalytic reduction of the product obtained in step a) and subsequent precipitation with sulfuric acid.

| | |
|---|---|
| Yield: | 28.3 g(94.1% of the theoretical) |

1.3.5. Preparation of N-(2,4-diaminophenyl)pyrrolidine sulfate

Step a) N-(2,4-dinitrophenyl)pyrrolidine

Step a) was carried out in the same way as Example 1.3.5. step a) by reacting 2,4-dinitrochlorobenzene with pyrrolidine.

| | |
|---|---|
| Yield: | 21.5 g(89.9% of the theoretical) |
| Melting point: | 79-81° C. |
| IR: | 3122 cm⁻¹(v CH_{Ar}), 2990, 2956 cm⁻¹(v CH), 1612 |
| | cm⁻¹(v C═C), 1527, 1506 cm⁻¹(vₐₛ NO₂), 1327 cm⁻¹(vₛ |
| | NO₂). |
| ¹H-NMR: | 8.58 ppm(H³, d, ⁴J_{H,H}=2.72 Hz); 8.21 ppm(H⁵, dd, |
| | ³J_{H,H}=9.58 Hz, ⁴J_{H,H}=2.67 Hz); 7.17 ppm(H⁶, d, |
| | ³J_{H,H}=9.59 Hz); 3.31 ppm(4H, s, NC+E,uns H₂), 2.57 ppm(4H, |
| | s, NCH₂C+E,uns H₂). |

Step b) N-(2,4-diaminophenyl)pyrrolidine sulfate

Step b) was carried out in the same way as Example 1.3.1. step b) by catalytic reduction of the product obtained in step a) and subsequent precipitation with sulfuric acid.

| | |
|---|---|
| Yield: | 11.8 g(37.8% of the theoretical) |

1.3.6. Preparation of 2,4-diamino-N,N-di-(2-hydroxyethyl)aniline sulfate

Step a) 2,4-dinitro-N,N-di-(2-hydroxyethyl)aniline

Step a) was carried out in the same way as Example 1.3.1. step a) using 2,4-dinitrochlorobenzene and diethanolamine.

| | |
|---|---|
| Yield: | 22.0 g(80.5% of the theoretical) |
| Melting point: | 90-92° C. |
| IR: | 3076 cm⁻¹(v CH_{Ar}), 2927 cm⁻¹(v CH), 1606 cm⁻¹(v |
| | C═C), 1527, 1505 cm⁻¹(Vₐₛ NO₂), 1328 cm⁻¹(v₅ NO₂). |
| ¹H-NMR: | 8.55 ppm(H³, d, ⁴J_{H,H}=2.84 Hz); 8.22 ppm(H⁵, dd, |
| | ³J_{H,H}=9.57 Hz, ⁴J_{H,H}=2.85 Hz); 7.50 ppm(H⁶, d, |
| | ³J_{H,H}=9.59 Hz); 4.82 ppm(2H, t, ³J_{H,H}=5.27 Hz, OH); |
| | 3.60 ppm(4H, q, ³J_{H,H}=5.56 Hz, C+E,uns H₂OH); 3.54 ppm(4H, |
| | t, ³J_{H,H}=5.76 Hz NC+E,uns H₂). |

Step b) 2,4-diamino-N, N-di-(2-hydroxyethyl)aniline sulfate

Step b) was carried out in the same way as Example 1.3.1. step b) by catalytic reduction of the product obtained in step a) and subsequent precipitation with sulfuric acid.

| | |
|---|---|
| Yield: | 10.3 g(32.1% of the theoretical) |

1.3.7. Preparation of 2,4-diamino-N-(2-hydroxyethyl)-N-ethylaniline sulfate

Step a) 2,4-dinitro-N-(2-hydroxyethyl)-N-ethyl aniline

Step a) was carried out in the same way as Example 1.3.1. step a) by reacting 2,4-dinitrochlorobenzene with N-methyl ethanolamine.

| | |
|---|---|
| Yield: | 20.9 g(81.2% of the theoretical) |
| Melting point: | 105-108° C. |
| IR: | 3096 cm⁻¹(v CH_{Ar}), 2932, 2818 cm⁻¹(v CH), 1621 |
| | cm⁻¹(v C═C), 1582, 1523 cm⁻¹(Vₐₛ NO₂), 1342 cm⁻¹(vₛ |
| | NO₂). |
| ¹H-NMR | 8.86 ppm(H³, d, ⁴J_{H,H}=2.74 Hz); 8.26 ppm(H⁵, dd, |
| | ³J_{H,H}=9.58 Hz, ⁴J_{H,H}=2.73 Hz); 7.26 ppm(H⁶, d, |
| | ³J_{H,H}=9.66 Hz); 4.54 ppm(1H, s, OH); 3.53 ppm(2H, t, |
| | ³J_{H,H}=5.02 Hz, C+E,uns H₂OH); 3.51 ppm(2H, q, ³J_{H,H}=6.05 |
| | Hz, NC+E,uns H₂CH₃); 2.87 ppm(4H, t, ³J_{H,H}=6.11 Hz, |
| | NC+E,uns H₂CH₂OH); 2.65 ppm(3H, t, ³J_{H,H}=5.75 Hz, |
| | NCH₂C+E,uns H₃). |

Step b) 2,4-diamino-N-(2-hydroxyethyl)-N-ethyl aniline sulfate

Step a) was carried out in the same way as Example 1.3.1. step b) by catalytic reduction of the product obtained in step a) and subsequent precipitation with sulfuric acid.

| | |
|---|---|
| Yield: | 8 g(34.6% of the theoretical). |

1.3.8. Preparation of N-(2,4-diaminophenyl)azepan sulfate

Step a) N-(4-amino-2-nitrophenyl)azepan

4-Amino-2-nitrochlorobenzene is reacted with azepan in the same way as in Example 1.3.1. step a).

| | |
|---|---|
| Yield: | 37.2 g(39.5% of the theoretical) |
| Melting point: | 72-73.5° C. |
| IR: | 3467, 3380 cm⁻¹(v CH_{Ar}), 2926, 2853 cm⁻¹(v CH), 1631 |
| | cm⁻¹(v C═C), 1520(Vₐₛ NO₂), 1360 cm⁻¹(vₛ NO₂). |
| ¹H-NMR: | 7.07 ppm(H⁶, d, ³J_{H,H}=8.64 Hz); 6.79 ppm(H⁵, dd, |
| | ³J_{H,H}=6.96 Hz, ⁴J_{H,H}=2.59 Hz); 6.75 ppm(H³, d, |
| | ⁴J_{H,H}=2.68 Hz); 5.24 ppm(2H, s, NH₂); 3.00 ppm(4H, t, |
| | ³J_{H,H}=5.49 Hz, NC+E,uns H₂); 1.62 . . . 1.56 ppm(8H, m, |
| | NCH₂C+E,uns H₂C+E,uns H₂C+E,uns H₂C+E,uns H₂). |

Step b) N-(2,4-diaminophenyl)azepan sulfate

The product was prepared by catalytic reduction of the product obtained in step a) and subsequent precipitation with sulfuric acid.

| | |
|---|---|
| Yield: | 19.5 g(64.5% of the theoretical) |

1.3.9. Preparation of 4-(2-hydroxyethylamino)-2-amino-N,N-dimethyl aniline sulfate

Step a) 4-Amino-2-nitro-N,N-dimethyl aniline

62.5 g (0.4 mole) of 4-fluoro-3-nitraniline, 45.1 g (0.4 mole, 40% solution) of dimethyl amine and 21.2 g (0.2 mole) of sodium carbonate were introduced into 250 ml of 1,2-dimethoxyethane. The mixture was heated under reflux until the conversion was complete, after which 1 g of active carbon and 1 g of Celite were added and, after stirring for about 30 minutes, the reaction mixture was filtered. The product was freed from the solvent in a rotary evaporator and the oil obtained was further processed.

| | |
|---|---|
| Yield: | 62.8 g(86.6% of the theoretical) |
| Melting point: | (oil) |
| IR: | 3373 cm⁻¹(v CH_{Ar}), 3227, 2946, 2870, 2792 cm⁻¹(v |
| | CH), 1631 cm⁻¹(v C═C), 1564, 1525 cm⁻¹(Vₐₛ NO₂), |
| | 1353, 1293 cm⁻¹(vₛ NO₂). |
| ¹H-NMR: | 7.10 ppm(H⁶, d, ³J_{H,H}=8.77 Hz); 6.94 ppm(H³, d, |
| | ⁴J_{H,H}=2.64 Hz); 6.83 ppm(H⁵, dd, ³J_{H,H}=8.77 Hz, |
| | ⁴J_{H,H}=2.70 Hz); 5.29 ppm(2H, s, N+E,uns H₂); 2.59 ppm(6H, s, |
| | N(C+E,uns H₃)₂). |

Step b) 4-(2-chloroethoxycarbonylamino)-2-nitro-N,N-dimethyl aniline

28.5 g (160 mmoles) of 4-amino-2-nitro-N,N-dimethyl aniline and 9 g (80 mmoles) of calcium carbonate were introduced into 100 ml of 1,2-dimethoxyethane. 22.5 g (160 mmoles) of chloroformic acid-2-chloroethyl ester were added dropwise to the solution at room temperature, after which the mixture was stirred until the reaction was complete. The pH value was then adjusted to 3-4 with concentrated hydrochloric acid and 100 g of an ice/water mixture were added. The product precipitated was filtered off under suction and washed twice with 100 ml of water.

| | |
|---|---|
| Yield: | 25.5 g(59.1% of the theoretical) |
| Melting point: | 178-180° C. |
| IR: | 3424 cm⁻¹(v CH_{Ar}), 3177, 3032 cm⁻¹(v CH), 1727 |
| | cm⁻¹(v C═O), 1607 cm⁻¹(C═C), 1544 cm⁻¹(Vₐₛ NO₂), |
| | 1322 cm⁻¹(vₛ NO₂), 1227 cm⁻¹(v O—C). |
| ¹H-NMR: | 8.03 ppm(H³, d, ⁴J_{H,H}=2.15 Hz); 7.62 ppm(H⁵, dd, |
| | ³J_{H,H}=9.06 Hz, ⁴J_{H,H}=2.55 Hz); 7.35 ppm(H⁶, d, |
| | ³J_{H,H}=9.11 Hz); 4.36 ppm (2H, q, C(O)OC+E,uns H₂); 3.89 |
| | ppm(2H, q, CH₂Cl); 2.83 ppm(6H, s, N(C+E,uns H₃)₂). |

Step c) 4-(2-hydroxyethylamino)-2-nitro-N,N-dimethylaniline

23.0 g (80 mmoles) of 4-(2-chloroethoxycarbonylamino)-2-nitro-N,N-dimethyl aniline were introduced into 100 ml of ethanol. 33 9 (412 mmoles) of 50% sodium hydroxide solution were added dropwise to the solution at room temperature, after which the mixture was stirred until the conversion was complete. The mixture was then heated to about 80° C. and the solution was filtered. The filtrate was then adjusted to pH 8 with acetic acid. 75 ml of water were then added and ethanol was distilled off until the boiling temperature was 99° C. After cooling, the solution was extracted with ethyl acetate, the ester phase was dried over sodium sulfate and the solution was concentrated in vacuo. The product crystallized after rubbing with a glass rod. It was filtered off under suction and dried.

| | |
|---|---|
| Yield: | 12.1 g(67.2% of the theoretical) |
| Melting point: | 110° C. |
| IR: | 3285 cm⁻¹(v CH_{Ar}), 3178 cm⁻¹(v CH), 1640 cm⁻¹(v |
| | C═O), 1559 cm⁻¹(Vₐₛ NO₂), 1413 cm⁻¹(vₛ NO₂), 810 |
| | cm⁻¹(v CHₒₒₚ). |
| ¹H-NMR: | 7.19 ppm(H⁶, d, ³J_{H,H}=9.60 Hz); 6.855 ppm(H⁵, dd, |
| | ³J_{H,H}=9.64 Hz, ⁴J_{H,H}=2.87 Hz); 6.854 ppm(H³, d, |
| | ⁴J_{H,H}=2.50 Hz); 6.01 ppm(1H, t, ³J_{H,H}=11 Hz, CH₂O+E,uns H); |
| | 3.54 ppm(2H, t, ³J_{H,H=5.81 Hz, NCH}₂); 3.07 ppm(2H, q, |
| | ³J_{H,OH}=11.35 Hz, ³J_{H,H}=5.65 Hz, C+E,uns H₂OH); 2.59 |
| | ppm(6H, s, N(C+E,uns H₃)₂). |

Step d) 4-(2-hydroxyethylamino)-2-amino-N,N-dimethyl aniline sulfate

100 ml of methanol were introduced into a 0.3 liter autoclave, 12.0 g (53 mmoles) of 4-(2-hydroxyethylamino)-2-nitro-N,N-dimethyl aniline (from step c) were dissolved therein and 2 g of palladium on active carbon 10% (Degussa) were added. After the autoclave had been closed and blanketed with nitrogen, the mixture was hydrogenated under a pressure of 4 bar and at a temperature of 35-40° C. until no more hydrogen was taken up. 1.3 g of active carbon was added under nitrogen to the warm solution and the catalyst was filtered off. 7 g of 80% sulfuric acid (alternatively: 16 ml of 35% hydrochloric acid per 0.1 mole) were added dropwise to the solution while cooling with ice at 5° C. The product precipitated was filtered off under suction, washed with methanol and dried.

| | |
|---|---|
| Yield: | 12.2 g(78.8% of the theoretical) |
| Melting point: | >250° C. |
| IR: | 3397 cm⁻¹(v OH), 3234 cm⁻¹(v CH_{Ar}), 2920 cm⁻¹(v |
| | CH_{Alkyl}), 1661, 1630, 1515 cm⁻¹(v NH₃⁺), 1596 cm⁻¹(v |
| | C═C). |
| ¹H-NMR: | 7.65-4.75 ppm(6H, NH₃⁺); 6.65 ppm(H⁶, d, |
| | ³J_{H,H}=8.34 Hz); 6.51 ppm(H⁵, dd, ³J_{H,H}=8.48 Hz, |
| | ⁴J_{H,H}=2.34 Hz); 6.46 ppm(H³, d, ⁴J_{H,H}=2.29 Hz); 2.77 |
| | ppm(6H, s, N(CH₃)₂). |

1.3.10. Preparation of N-[4-(2-hydroxyethylamino)-2-aminophenyl]morpholine sulfate

Step a) N-(4-amino-2-nitrophenyl)morpholine

Step a) was carried out in the same way as Example 1.3.9. step a) by reacting 4-fluoro-3-nitraniline with morpholine.

| | |
|---|---|
| Yield: | 61.9 g(69.3% of the theoretical) |
| Melting point: | 131-132° C. |
| IR: | 3479 cm⁻¹(v CH_{Ar}), 3042, 2958, 2857, 2830 cm⁻¹(v |
| | CH), 1626 cm⁻¹(v C═C), 1515(vₐₛ NO₂), 1343 cm⁻¹(vₛ |
| | NO₂). |
| ¹H-NMR: | 7.18 ppm(H⁶, d, ³J_{H,H}=8.66 Hz); 6.88 ppm(H³, d, |
| | ⁴J_{H,H}=2.44 Hz); 6.80 ppm(H⁵, dd, ³J_{H,H}=8.59 Hz, |
| | ⁴J_{H,H}=2.43 Hz); 5.46 ppm(2H, s, N+E,uns H₂); 3.63 ppm(4H, t, |
| | ³J_{H,H}=4.66 Hz, OCH₂); 2.79 ppm(4H, t, ³J_{H,H}=4.43 Hz, |
| | NCH₂). |

Step b) N-[4-(2-chloroethoxycarbonylamino)-2-nitrophenyl]morpholine

Step b) was carried out in the same way as Example 1.3.9. step b) by reacting N-(4-amino-2-nitrophenyl)morpholine with chloroformic acid-2-chloroethyl ester.

| | |
|---|---|
| Yield: | 32.7 g(95.1% of the theoretical) |
| Melting point: | 121-122° C. |
| IR: | 3304 cm⁻¹(v CH_{Ar}), 3177, 3102 cm⁻¹(v CH), 1732 |
| | cm⁻¹(v C═O), 1596 cm⁻¹(C═C), 1537 cm⁻¹(vₐₛ NO₂), |
| | 1342 cm⁻¹(vₛ NO₂), 1224 cm⁻¹(v O—C). |
| ¹H-NMR: | 10.1 ppm(1H, s, NH); 8.01 ppm(H⁶, d, ³J_{H,H}=2.21 Hz); |
| | 7.64 ppm(H⁵, dd, ³J_{H,H}=8.89 Hz, ⁴J_{H,H}=2.46 Hz); 7.85 |
| | ppm(H³, d, ³J_{H,H}=8.94 Hz); 4.38 ppm(2H, t, ³J_{H,H}=5.16 |
| | Hz, C(O)OC+E,uns H₂); 3.89 ppm(2H, t, ³J_{H,H}=5.16 Hz, |
| | CH₂Cl); 3.69 ppm(4H, t, ³J_{H,H}=4.48 Hz, CH₂OCH₂); |
| | 2.92 ppm(4H, t, ³J_{H,H}=4.50 Hz, CH₂NCH₂). |

Step c) N-[4-(2-hydroxyethylamino)-2-nitrophenyl]morpholine

Step c) was carried out in the same way as Example 1.3.9. step c) by reacting N-[4-(2-chloroethoxycarbonylamino)-2-nitrophenyl]morpholine with potassium hydroxide.

| | |
|---|---|
| Yield: | 20 g(93.5% of the theoretical) |
| Melting point: | (oil) |
| IR: | 3282 cm⁻¹(v CH_{Ar}), 2941 cm⁻¹(v CH), 1632 cm⁻¹(v |
| | C═O), 1567 cm⁻¹(vₐₛ NO₂), 1368 cm⁻¹(vₛ NO₂). |
| ¹H-NMR: | 8.6 ppm(4H, s, NH/OH); 7.36 ppm(H⁶, d, ³J_{H,H}=8.63 |
| | Hz); 7.21 ppm(H³, d, ⁴J_{H,H}=2.19 Hz); 7.10 ppm(H⁵, dd, |
| | ³J_{H,H}=8.59 Hz, ⁴J_{H,H}=2.19 Hz); 3.84 ppm(4H, t, |
| | ³J_{H,H}=4.22 Hz, CH₂OCH₂); 3.67 ppm(2H, t, ³J_{H,H}=5.47 |
| | Hz, C+E,uns H₂OH); 3.23 ppm(2H, t, ³J_{H,H}=5.48 Hz, |
| | NC+E,uns H₂CH₂OH); 2.97 ppm(4H, t, ³J_{H,H}=4.06 Hz, |
| | CH₂NCH₂). |

Step d) N-[4-(2-hydroxyethylamino)-2-aminophenyl]morpholine sulfate

Step d) was carried out in the same way as Example 1.3.9. step d) by catalytic reduction of the product obtained in step c) and subsequent precipitation with sulfuric acid.

| | |
|---|---|
| Yield: | 7.4 g(28.3% of the theoretical) |

1.3.11. Preparation of N-[4-(2-hydroxyethylamino)-2-aminophenyl]piperidine sulfate

Step a) N-(4-amino-2-nitrophenyl)piperidine

Step a) was carried out in the same way as Example 1.3.9. step a) by reaction of 4-fluoro-3-nitraniline with piperidine.

| | |
|---|---|
| Yield: | 80.6 g(91.1% of the theoretical) |
| Melting point: | 112-113° C. |
| IR: | 3486, 3389 cm⁻¹(v CH_{Ar}), 2950, 2934, 2848 cm⁻¹(v |
| | CH), 1629 cm⁻¹(v C═C), 1511(vₐₛ NO₂), 1361 cm⁻¹(vₛ |
| | NO₂). |
| ¹H-NMR: | 7.12 ppm(H⁶, d, ³J_{H,H}=8.68 Hz); 6.86 ppm(H³, d, |
| | ⁴J_{H,H}=2.51 Hz); 6.78 ppm(H⁵, dd, ³J_{H,H}=8.60 Hz, |
| | ⁴J_{H,H}=2.52 Hz); 5.38 ppm(2H, s, N+E,uns H₂); 2.75 ppm(4H, t, |
| | ³J_{H,H}=4.66 Hz, NCH₂), 1.54 . . . 1.45 ppm(6H, m, |
| | NCH₂+E,uns CH₂CH₂CH₂. |

Step b) N-[4-(2-chloroethoxycarbonylamino)-2-nitrophenyl]piperidine

Step b) was carried out in the same way as Example 1.3.9. step b) by reacting N-(4-amino-2-nitrophenyl)piperidine with chloroformic acid-2-chloroethyl ester.

| | |
|---|---|
| Yield: | 31.1 g(94.8% of the theoretical) |
| Melting point: | 74-76° C. |
| IR: | 3373 cm⁻¹(v CH_{Ar}), 2936, 2853 cm⁻¹(v CH), 1730 |
| | cm⁻¹(v C═O), 1588 cm⁻¹(C═C), 1532 cm⁻¹(vₐₛ NO₂), |
| | 1307 cm⁻¹(vₛ NO₂), 1224 cm⁻¹(v O—C). |
| ¹H-NMR: | 10.0 ppm(1H, s, NH); 7.96 ppm(H⁶, d, ³J_{H,H}=2.35 Hz); |
| | 7.59 ppm(H⁵, dd, ³J_{H,H}=8.95 Hz, ⁴J_{H,H}=2.00 Hz); 7.31 |
| | ppm(H³, d, ³J_{H,H}=8.97 Hz); 4.36 ppm(2H, t, ³J_{H,H}=5.19 |
| | Hz, C(O)OC+E,uns H₂); 3.88 ppm(2H, t, ³J_{H,H}=5.19 Hz, |
| | CH₂Cl); 2.87 ppm(4H, t, ³J_{H,H}=5.03 Hz, CH₂NCH₂; |
| | 1.59 . . . 1.50 ppm(6H, m, CH₂CH₂CH₂). |

Step c) N-[4-(2-hydroxyethylamino)-2-nitrophenyl]piperidine

Step c) was carried out in the same way as Example 1.3.9. step c) by reacting N-[4-(2-chloroethoxycarbonylamino)-2-nitrophenyl]piperidine with sodium hydroxide.

| | |
|---|---|
| Yield: | 19 g(90% of the theoretical) |
| Melting point: | (oil) |

Step d) N-[4-(2-hydroxyethylamino)-2-aminophenyl]piperidine sulfate

Step d) was carried out in the same way as Example 1.3.9. step d) by catalytic reduction of the product obtained in step c) and subsequent precipitation with sulfuric acid.

| | |
|---|---|
| Yield: | 11.4 g(83.5% of the theoretical) |
| Melting point: | 200-202° C. |
| IR: | 3413 cm⁻¹(v OH), 3197 cm⁻¹(v CH_{Ar}), 2946 cm⁻¹(v |
| | CH), 1628 cm⁻¹(v C═C), 1516 cm⁻¹(vₐₛ NO₂), 1451 |
| | cm⁻¹(vₛ NO₂). |
| ¹H-NMR: | 7.6 ppm(6H, s, NH₃⁺/NH₂⁺/OH); 7.48 ppm(H⁶, d, |
| | ³J_{H,H}=8.89 Hz); 6.87 ppm(H³, d, ⁴J_{H,H}=2.09 Hz); 6.75 |
| | ppm(H⁵, dd, ³J_{H,H}=8.83 Hz, ⁴J_{H,H}=2.09 Hz); 3.64 |
| | ppm(2H, t, ³J_{H,H}=5.55 Hz, CH₂OH); 3.46 ppm(4H, t, |
| | ³J_{H,H}=4.58 Hz, CH₂NCH₂); 3.20 ppm(2H, t, ³J_{H,H}=5.54 |
| | Hz, NC+E,uns H₂CH₂OH); 2.06 ppm(4H, s, |
| | C+E,uns H₂CH₂NCH₂C+E,uns H₂); 1.65 ppm(4H, s, |
| | CH₂CH₂C+E,uns H₂CH₂CH₂). |

1.3.12. Preparation of N-[4-(3-hydroxypropylamino)-2-aminophenyl]morpholine dihydrochloride

Step a) N-(4-amino-2-nitrophenyl)morpholine

Step a) was carried out by reacting 4-fluoro-3-nitraniline with morpholine as described in Example 1.3.10. step a).

Step b) N-[4-(3-chloropropoxycarbonylamino)-2-nitrophenyl]morpholine

The compound was prepared by reacting N-(4-amino-2-nitrophenyl)-morpholine with chloroformic acid-3-chloropropyl ester as in Example 1.3.10. step b).

| | |
|---|---|
| Yield: | 32.7 g(95.1% of the theoretical) |
| Melting point: | 122-124° C. |
| IR: | 3245 cm⁻¹(v CH_{Ar}), 2964 cm⁻¹(v CH), 1737 cm⁻¹(v |
| | C═O), 1596 cm⁻¹(C═C), 1537 cm⁻¹(vₐₛ NO₂), 1373 |
| | cm⁻¹(vₛ NO₂), 1221 cm⁻¹(v O—C). |
| ¹H-NMR: | 9.95 ppm(1H, s, NH); 7.99 ppm(H³, s); 7.62 ppm(H⁵, |
| | dd, ³J_{H,H}=8.81 Hz, ⁴J_{H,H}=1.81 Hz); 7.38 ppm(H⁶, d, |
| | ³J_{H,H}=8.93 Hz); 4.23 ppm(2H, t, ³J_{H,H}=6.18 Hz, |
| | C(O)OC+E,uns H₂); 3.75 ppm(2H, t, ³J_{H,H}=6.43 Hz, CH₂Cl); |
| | 3.69 ppm(4H, t, ³J_{H,H}=4.08 Hz, CH₂OCH₂); 2.93 |
| | ppm(4H, t, ³J_{H,H}=4.06 Hz, CH₂NCH₂); 2.10 ppm(2H, q, |
| | ³J_{H,H}=6.27 Hz, CH₂C+E,uns H₂CH₂). |

Step c) N-[4-(3-hydroxypropylamino)-2-nitrophenyl]morpholine

The compound was prepared by reacting N-[4-(3-chloropropoxycar-bonylamino)-2-nitrophenyl]morpholine with sodium hydroxide as in Example 1.3.10. step c).

| | |
|---|---|
| Yield: | 20.4 g(90.5% of the theoretical) |
| Melting point: | 93-95° C. |
| IR: | 3433, 3347 cm⁻¹(v CH_{Ar}), 2964, 2867 cm⁻¹(v CH), 1622 |
| | cm⁻¹(v C═C), 1543 cm⁻¹(vₐₛ NO₂), 1371 cm⁻¹(vₛ NO₂). |
| ¹H-NMR: | 7.24 ppm(H⁶, d, ³J_{H,H}=8.55 Hz); 6.84 ppm(H³, d, |
| | ⁴J_{H,H}=2.52 Hz); 6.81 ppm(H⁵, dd, ³J_{H,H}=8.59 Hz, |
| | ⁴J_{H,H}=2.72 Hz); 6.03 ppm(1H, t, ³J_{H,H}=5.46 Hz, OH); |
| | 3.63 ppm(4H, t, ³J_{H,H}=4.49 Hz, CH₂OCH₂); 3.49 |
| | ppm(2H, t, ³J_{H,H}=6.21 Hz, C+E,uns H₂OH); 3.06 ppm(2H, q, |
| | ³J_{H,H}=6.75 Hz, ³J_{H,NH}=12.53 Hz, |
| | NC+E,uns H₂CH₂—CH₂OH); 2.81 ppm(4H, t, ³J_{H,H}=4.49 Hz, |
| | CH₂NCH₂); 1.67 ppm(2H, qi, ³J_{H,H}=6.56 Hz, |
| | NCH₂C+E,uns H₂CH₂OH). |

Step d) N-[4-(3-hydroxypropylamino)-2-aminophenyl]morpholine dihydrochloride

The product was obtained by catalytic reduction of the product obtained in step c) and salt formation with hydrochloric acid.

| | |
|---|---|
| Yield: | 11.4 g(83.5% of the theoretical) |
| Melting point: | 139-141° C. |
| IR: | 3365, 3196 cm⁻¹(v CH_{Ar}), 2614, 2436 cm⁻¹(v NH₃⁺, |
| | NH₂⁺), 1628 cm⁻¹(v C═C), 1123 cm⁻¹(v C—O—C). |
| ¹H-NMR: | 7.39 ppm(H⁶, d, ³J_{H,H}=8.62 Hz); 7.33 ppm(H³, d, |
| | ⁴J_{H,H}=2.14 Hz); 7.21 ppm(H⁵, dd, ³J_{H,H}=8.54 Hz, |
| | ⁴J_{H,H}=2.09 Hz); 3.83 ppm(4H, t, ³J_{H,H}=4.15 Hz, |
| | CH₂OCH₂); 3.48 ppm(2H, t, ³J_{H,H}=6.04 Hz, C+E,uns H₂OH); |
| | 3.22 ppm(2H, q, ³J_{H,H}=7.68 Hz, NC+E,uns H₂—CH₂CH₂OH)); |
| | 2.97 ppm(4H, s, CH₂NCH₂); 1.83 ppm(2H, qi, ³J_{H,} |
| | H=6.84 Hz, NCH₂C+E,uns H₂CH₂OH). |

1.3.13. Preparation of N-[4-(3-hydroxypropylamino)-2-aminophenyl]piperidine sulfate

Step a) N-(4-amino-2-nitrophenyl)piperidine

Step a) was carried out in the same way as Example 1.3.9. step a) by reacting 4-fluoro-3-nitraniline with piperidine.

Step b) N-[4-(3chloropropoxycarbonylamino)-2-nitrophenyl]piperidine

Step b) was carried out in the same way as Example 1.3.9. step b) by reacting N-(4-amino-2-nitrophenyl)piperidine with chloroformic acid-3-chloropropyl ester.

| | |
|---|---|
| Yield: | 20.6 g(60.4% of the theoretical) |
| Melting point: | (oil) |
| IR: | 3331 cm⁻¹(v CH_{Ar}), 2938, 2854 cm⁻¹(v CH), 1708 |
| | cm⁻¹(v C═O), 1587 cm⁻¹(C═C), 1532 cm⁻¹(vₐₛ NO₂), |
| | 1305 cm⁻¹(vₛ NO₂), 1224 cm⁻¹(v O—C). |
| ¹H-NMR: | 9.95 ppm(1H, s, NH); 7.95 ppm(H⁶, d, ³J_{H,H}=1.71 Hz); |
| | 7.57 ppm(H⁵, dd, ³J_{H,H}=8.87 Hz, ⁴J_{H,H}=2.05 Hz); 7.28 |
| | ppm(H³, d, ³J_{H,H}=8.89 Hz); 4.21 ppm(2H, t, ³J_{H,H}=6.19 |
| | Hz, C(O)OC+E,uns H₂); 3.73 ppm(2H, t, ³J_{H,H}=6.44 Hz, |
| | CH₂Cl); 2.85 ppm(4H, t, ³J_{H,H}=4.83 Hz, CH₂NCH₂); |
| | 1.57 ppm(4H, m, C+E,uns H₂CH₂C+E,uns H₂); 1.49 ppm(2H, m, |
| | CH₂C+E,uns H₂CH₂). |

Step c) N-[4-(3-hydroxypropylamino)-2-nitrophenyl]piperidine

Step c) was carried out in the same way as Example 1.3.9. step c) by reacting N-[4-(3-chloropropoxycarbonylamino)-2-nitrophenyl]piperidine with sodium hydroxide.

Yield: 20.6 g (60.4% of the theoretical)

Step d) N-[4-(3-hydroxypropylamino)-2-aminophenyl]piperidine sulfate

Step d) was carried out in the same way as Example 1.3.9. step d) by catalytic reduction of the product obtained in step c) and subsequent precipitation with sulfuric acid.

Yield: 20.6 g (60.4% of the theoretical)

1.3.14. Preparation of N-[4-(3-hydroxypropylamino)-2-aminophenyl]pyrrolidine sulfate

Step a) N-(4-amino-2-nitrophenyl)pyrrolidine

Step a) was carried out in the same way as Example 1.3.9. step a) by reacting 4-fluoro-3-nitraniline with pyrrolidine.

| | |
|---|---|
| Yield: | 61.0 g(73.6% of the theoretical) |
| Melting point: | 84-86° C. |
| IR: | 3486, 3389 cm⁻¹(v CH_{Ar}), 2950, 2934, 2848 cm⁻¹(v |
| | CH), 1624 cm⁻¹(v C═C), 1521(vₐₛ NO₂), 1364 cm⁻¹(vₛ |
| | NO₂). |
| ¹H-NMR: | 6.98 ppm(H³, d, ⁴J_{H,H}=2.28 Hz); 6.87 ppm(H⁵, H⁶, m); |
| | 4.92 ppm(2H, s, N+E,uns H₂); 3.00 ppm(4H, t, ³J_{H,H}=6.49 Hz, |
| | NCH₂); 1.85 ppm(6H, m, NCH₂+E,uns CH₂CH₂). |

Step b) N-[4-(3-chloropropoxycarbonylamino)-2-nitrophenyl]pyrrolidine

Step b) was carried out in the same way as Example 1.3.9. step b) by reacting N-(4-amino-2-nitrophenyl)-pyrrolidine with chloroformic acid-3-chloropropyl ester.

| | |
|---|---|
| Yield: | 23.7 g(72.2% of the theoretical) |
| Melting point: | (oil) |
| IR: | 3331 cm⁻¹(v CH_{Ar}), 2967, 2873 cm⁻¹(v CH), 1703 |
| | cm⁻¹(v C═O), 1578 cm⁻¹(C═C), 1533 cm⁻¹(vₐₛ NO₂), |
| | 1365 cm⁻¹(vₛ NO₂), 1226 cm⁻¹(v O—C). |
| ¹H-NMR: | 9.7 ppm(1H, s, NH); 7.96 ppm(H⁶, s); 7.49 ppm(H⁵, dd, |
| | ³J_{H,H}=9.07 Hz, ⁴J_{H,H}=2.04 Hz); 6.98 ppm(H³, d, |
| | ³J_{H,H}=9.24 Hz); 4.20 ppm(2H, t, ³J_{H,H}=6.22 Hz, |
| | C(O)OC+E,uns H₂); 3.73 ppm(2H, t, ³J_{H,H}=6.46 Hz, CH₂Cl); |
| | 3.09 ppm(4H, t, ³J_{H,H}=6.05 Hz, CH₂NCH₂); 2.08 |
| | ppm(2H, q, ³J_{H,H}=6.35 Hz, CH₂C+E,uns H₂CH₂Cl); 1.88 |
| | ppm(4H, m, ³J_{H,H}=6.27 Hz, CH₂CH₂). |

Step c) N-[4-(3-hydroxypropylamino)-2-nitrophenyl]pyrrolidine

Step c) was carried out in the same way as Example 1.3.9. step c) by reacting N-[4-(3-chloropropoxycarbonylamino)-2-nitrophenyl]pyrrolidine with sodium hydroxide.

| | |
|---|---|
| Yield: | 15.2 g(81.8% of the theoretical) |

Step d) N-[4-(3-hydroxypropylamino)-2-aminophenyl]pyrrolidine sulfate

Step d) was carried out in the same way as Example 1.3.9. step d) by catalytic reduction of the product obtained in step c) and subsequent precipitation with sulfuric acid.

| | |
|---|---|
| Yield: | 16.6 g(86.9% of the theoretical) |

1.3.15. Preparation of N-[2-amino-4-(3-hydroxypropylamino)phenyl]azepan sulfate

Step a) N-(4-amino-2-nitrophenyl)azepan

Step a) was carried out in the same way as Example 1.3.9. step a) by reacting 4-fluoro-3-nitraniline with azepan.

| | |
|---|---|
| Yield: | 37.2 g(39.5% of the theoretical) |
| Melting point: | 72-73.5° C. |
| IR: | 3467, 3380 cm⁻¹(v CH_{Ar}), 2926, 2853 cm⁻¹(v CH), 1631 |
| | cm⁻¹(v C═C), 1520(vₐₛ NO₂), 1360 cm⁻¹(vₛ NO₂). |
| ¹H-NMR: | 7.07 ppm(H⁶, d, ³J_{H,H}=8.64 Hz); 6.79 ppm(H⁵, dd, |
| | ³J_{H,H}=6.96 Hz, ⁴J_{H,H}=2.59 Hz); 6.75 ppm(H³, d, |
| | ⁴J_{H,H}=2.68 Hz); 5.24 ppm(2H, s, N+E,uns H₂); 3.00 ppm(4H, t, |
| | ³J_{H,H}=5.49 Hz, NCH₂); 1.62 . . . 1,56 ppm(8H, m, |
| | NCH₂C+E,uns H₂C+E,uns H₂C+E,uns H₂C+E,uns H₂ |

Step b) N-[4-(3-chloropropoxycarbonylamino)-2-nitrophenyl)azepan

Step b) was carried out in the same way as Example 1.3.9. step b) by reacting N-(4-amino-2-nitrophenyl)azepan with chloroformic acid-3-chloropropyl ester.

| | |
|---|---|
| Yield: | 13.6 g(63.7% of the theoretical) |
| Melting point: | 61-63° C. |
| IR: | 3395, 3098 cm⁻¹(v CH_{Ar}), 2928, 2856 cm⁻¹(v CH), 1726 |
| | cm⁻¹(v C═O), 1574 cm⁻¹(v C═C), 1533(vₐₛ NO₂), 1354 |
| | cm⁻¹(vₛ NO₂), 1226 cm⁻¹(v O—C). |
| ¹H-NMR: | 9.75 ppm(1H, s, NH); 7.90 ppm(H³, s); 7.48 ppm(H⁵, |
| | dd, ³J_{H,H}=9.08 Hz, ⁴J_{H,H}=1.89 Hz); 7.15 ppm(H⁶, d, |
| | ⁴J_{H,H}=9.17 Hz); 4.20 ppm, 4.23 ppm(2H, t, ³J_{H,H}=6.17 |
| | Hz, C(O)OC+E,uns H₂); 3.72 ppm(2H, t, ³J_{H,H}=6.44 Hz, |
| | CH₂Cl); 3.13 ppm(4H, t, ³J_{H,H}=5.44 Hz, CH₂NCH₂); |
| | 2.08 ppm(2H, m, OCH₂C+E,uns H₂CH₂Cl); 1.67 ppm(4H, s, |
| | NCH₂C+E,uns H₂); 1.49 ppm(4H, s, NCH₂CH₂C+E,uns H₂). |

Step c) N-[4-(3-hydroxypropylamino)-2-nitropheny]lazepan

Step c) was carried out in the same way as Example 1.3.9. step c) by reacting N-[4-(3-chloropropoxycarbonylamino)-2-nitrophenyl]azepan with sodium hydroxide.

| | |
|---|---|
| Yield: | 12.8 g(34.3% of the theoretical) |

Step d) N-[2-amino-4-(3-hydroxypropylamino)-phenyl]azepan sulfate

Step d) was carried out in the same way as Example 1.3.9. step d) by catalytic reduction of the product obtained in step c) and subsequent precipitation with sulfuric acid.

| | |
|---|---|
| Yield: | 12.9 g(81.8% of the theoretical) |

1.3.16. Preparation of N-{2-amino-4-[di-(2-hydroxyethyl)amino]phenyl}pyrrolidine sulfate

Step a) N-{2-nitro-4-[di-(2-hydroxyethyl)amino]phenyl}pyrrolidine

24.4 g (0.1 mole) of 4-fluoro-3-nitro-N,N-di-(2-hydroxyethyl)-aniline, 7.8 g (0.11 mole) of pyrrolidine, 5.3 g (0.05 mole) of potassium carbonate and 0.25 g of methyl tri(C₆₋₈)alkyl ammonium chloride (70% in isopropanol) were dissolved in 65 ml of 1,2-dimethoxyethane and the resulting solution was heated under reflux until the reaction was complete. The undissolved salts were filtered off in the heat and the mother liquor was cooled. The product precipitated was filtered off under suction and dried.

| | |
|---|---|
| Yield: | 23.8 g (80.6% of the theoretical) |
| IR: | 3401 cm⁻¹(v CH_{Ar}), 2965, 2870 cm⁻¹(v CH), 1630 cm⁻¹(v |
| | C═C), 1547(vₐₛ NO₂), 1358 cm⁻¹(vₛ NO₂). |
| ¹H-NMR: | 7.02 ppm(H³H⁵H⁶, m); 4.73 ppm(2H, t, ³J_{H,OH}=5.38 Hz, |
| | CH₂—O+E,uns H); 3.53 ppm(4H, q, ³J_{H,H}=6.34 Hz, CH₂OH); 3.36 |
| | ppm(4H, t, ³J_{H,H}=5.74 Hz, NC+E,uns H₂CH₂OH); 3.04 ppm(4H, t, |
| | ³J_{H,H}=6.39 Hz, NCH₂); 1.88 ppm(4H, t, ³J_{H,H}=6.42 Hz, |
| | NCH₂+E,uns CH₂CH₂). |

Step b) N-{2-amino-4-[di-(2-hydroxyethyl)amino[phenyl}pyrrolidine sulfate

Step b) was carried out in the same way as Example 1.3.9. step d) by catalytic reduction of the product obtained in step a) and subsequent precipitation with sulfuric acid.

| | |
|---|---|
| Yield: | 11.5 g (45.3% of the theoretical) |
| IR: | 3401 cm⁻¹(v CH_{Ar}), 2965, 2870 cm⁻¹(v CH), 1630 cm⁻¹(v |
| | C═C), 1547(vₐₛ NO₂), 1358 cm⁻¹(vₛ NO₂). |
| ¹H-NMR: | 7.02 ppm(H³H⁵H⁶, m); 4.73 ppm(2H, t, ³J_{H,OH}=5.38 Hz, |
| | CH₂—O+E,uns H); 3.53 ppm(4H, q, ³J_{H,H}=6.34 Hz, CH₂OH); 3.36 |
| | ppm(4H, t, ³J_{H,H}=5.74 Hz, NC+E,uns H₂CH₂OH); 3.04 ppm(4H, t, |
| | ³J_{H,H}=6.39 Hz, NCH₂); 1.88 ppm(4H, t, ³J_{H,H}=6.42 Hz, |
| | NCH₂+E,uns CH₂CH₂). |

1.3.17. Preparation of N-methyl-2,5-diaminoaniline sulfate

Step a) N-methyl-2-nitro-5-acetaminoaniline

100 ml of 1,2-dimethoxyethane, 21.5 g (0.1 mole) of 2-nitro-5-acetylaminochlorobenzene, 15.1 g (0.11 mole) of potassium carbonate and 12.4 g (0.11 mole) of 40% methylamine solution were introduced into an autoclave. The mixture was heated with stirring for 8 hours to 120° C. The inorganic salts were filtered off from the hot mixture and the solvent was distilled off in vacuo. The yellow powder precipitated was recrystallized from ethanol.

| | |
|---|---|
| Yield: | 17 g(100% of the theoretical) |
| IR: | 3351 cm⁻¹(v CH_{Ar}), 3303, 3183, 2808 cm⁻¹(v CH), 1694 |
| | cm⁻¹(v C═O), 1639 cm⁻¹(v C═C), 1582, 1562 cm⁻¹(vₐₛ |
| | NO₂), 1366, 1325 cm⁻¹(vₛ NO₂). |
| ¹H-NMR: | 10.2 ppm(1H, s, NHAc); 8.43 ppm(1H, m, NHCH₃); 8.19 |
| | ppm(H³, d, ³J_{H,H}=9.34 Hz); 7.56 ppm(H⁶, d, ⁴J_{H,H}=2.95 Hz); |
| | 6.97 ppm(H⁴, dd, ³J_{H,H}=9.42 Hz, ⁴J_{H,H}=2.14 Hz); 3.08 |
| | ppm(3H, t, ³J_{H,H}=4.93 Hz, NC+E,uns H₃); 2.27 ppm(3H, s, |
| | C(═O)CH₃). |

Step b) N-methyl-2-nitro-5-aminoaniline

15.9 g (0.095 mole) of N-methyl-5-acetamino-2-nitroaniline (from step a) were introduced into a mixture of 78 ml of water and 26 ml of methanol and the whole was heated under reflux for 1 hour with 25.8 ml of concentrated hydrochloric acid. On completion of the reaction, the mixture was adjusted to pH 7 with ammonia and the product was separated by stirring, filtered off under suction and washed with water.

| | |
|---|---|
| Yield: | 12.4 g(78.1% of the theoretical) |
| IR: | 3425 cm⁻¹(v CH_{Ar}), 3336, 3227, 2926 cm⁻¹(v CH), 1636 |
| | cm⁻¹(v C═C), 1577 cm⁻¹(vₐₛ NO₂), 1331 cm⁻¹(vₛ NO₂). |
| ¹H-NMR: | 8.33 ppm(1H, m, NHCH₃); 7.82 ppm(H³, d, ³J_{H,H}=9.37 Hz); |
| | 6.59 ppm(2H, s, NH₂); 6.0 ppm(H⁴, dd, ³J_{H,H}=9.36 Hz, |
| | ⁴J_{H,H}=2.12 Hz); 5.8 ppm(H⁶, d, ⁴J_{H,H}=2.11 Hz); 2.86 |
| | ppm(3H, t, ³J_{H,H}=4.96 Hz, NC+E,uns H₃). |

Step c) N-methyl-2,5-diaminoaniline sulfate

15.9 g (0.064 mole) of N-methyl-2-nitro-5-aminoaniline (from step b) were introduced into a 0.3 liter autoclave with 1 g of palladium on active carbon 10% (Degussa) in 180 ml of methanol. After the autoclave had been closed and blanketed with nitrogen, the mixture was hydrogenated under a pressure of 3 bar and at a temperature of 35 to 40° C. until no more hydrogen was taken up. 1.3 g of active carbon was added under nitrogen to the warm solution and the catalyst was filtered off. 8.4 g of 80% sulfuric acid were added dropwise to the solution while cooling with ice at 5° C. The product precipitated was filtered off under suction, washed with methanol and dried.

| | |
|---|---|
| Yield: | 14.4 g(90% of the theoretical) |
| Melting point: | >200° C. |
| IR: | 3384 cm⁻¹(v OH), 2878 cm⁻¹(v CH_{Ar}), 1602 cm⁻¹(v |
| | C═C). |
| ¹H-NMR: | 8.2-7.2 ppm(6H, NH₃⁺); 6.69 ppm(H³, d, |
| | ³J_{H,H}=7.82 Hz); 6.19 ppm(H⁴, s); 6.16 ppm(H⁶, s); 2.71 |
| | ppm(3H, s, NC+E,uns H₃). |

1.3.18. Preparation of N-ethyl-2,5diaminoaniline sulfate

Step a) N-ethyl-2-nitro-5-acetaminoaniline

Step a) was carried out in the same way as Example 1.3.17. step a) by reacting 2-nitro-5-acetylaminochlorobenzene with ethylamine.

| | |
|---|---|
| Yield: | 19.7 g(88.2% of the theoretical) of yellow crystals |
| IR: | 3343 cm⁻¹(v CH_{Ar}), 3225, 2971, 2873 cm⁻¹(v CH), 1702 |
| | cm⁻¹(v C═O), 1621 cm⁻¹(v C═C), 1582 cm⁻¹(vₐₛ NO₂), |
| | 1367 cm⁻¹(vₛ NO₂). |
| ¹H-NMR: | 10.31 ppm(1H, s, NHAc); 8.19 ppm(1H, t, NHEt, ³J_{H,H}=5.22 |
| | Hz); 8.02 ppm(H³, d, ³J_{H,H}=9.42 Hz); 7.46 ppm(H⁶, d, ⁴J_{H,H}= |
| | 1.97 Hz); 6.79 ppm(H⁴, dd, ³J_{H,H}=9.39 Hz, ⁴J_{H,H}=2.01 Hz); |
| | 3.08 ppm(2H, m, ³J_{H,CH3}=5.71 Hz, ³J_{CH2,CH3}=6.99 Hz, |
| | NC+E,uns H₂); 1.25 ppm(3H, t, ³J_{H,H}=7.11 Hz, NCH₂C+E,uns H₃). |

Step b) N-ethyl-2-nitro-5-aminoaniline

Step b) was carried out in the same way as Example 1.3.17. step b) by reacting N-ethyl-2-nitro-5-acetaminoaniline with hydrochloric acid.

| | |
|---|---|
| Yield: | 15.3 g(84.4% of the theoretical) |
| IR: | 3438 cm⁻¹(v CH_{Ar}), 3339, 3232, 2979 cm⁻¹(v CH), 1619 |
| | cm⁻¹(v C═C), 1564 cm⁻¹(vₐₛ NO₂), 1354 cm⁻¹(vₛ NO₂). |
| ¹H-NMR: | 8.29 ppm(1H, s, NH); 7.82 ppm(H³, d, ³J_{H,H}=9.22 Hz); 6.59 |
| | ppm(2H, s, NH₂); 6.0 ppm(H⁴, dd, ³J_{H,H}=9.48 Hz, ⁴J_{H,H}=2.0 |
| | Hz); 5.84 ppm(H⁶, d, ⁴J_{H,H}=2.03 Hz); 3.2 ppm(2H, m, |
| | ³J_{H,CH3}=5.45 Hz, ³J_{CH2,CH3}=7.08 Hz, NC+E,uns H₂); 1.24 ppm(3H, t, |
| | ³J_{CH2,CH3}=7.12 Hz, NCH₂C+E,uns H₃). |

Step c) N-ethyl-2,5diaminoaniline sulfate

Step c) was carried out in the same way as Example 1.3.17. step c) by catalytic reduction of N-ethyl-2-nitro-5-aminoaniline.

| | |
|---|---|
| Yield: | 15.4 g(85.8% of the theoretical) |
| Melting point: | >200° C. |
| IR: | 3369 cm⁻¹(v OH), 2883 cm⁻¹(v CH_{Ar}), 1618 cm⁻¹(v |
| | C═C). |
| ¹H-NMR: | 8.2-7.2 ppm(6H, NH₃⁺); 6.81 ppm(H³, s); 6.71 |
| | ppm(H⁴H⁶, s); 3.03 ppm(2H, q, J_{H,H}=7.11 Hz, NCH₂); |
| | 2.71 ppm(3H, t, J_{H,H}=7.11 Hz, NCH₂C+E,uns H₃). |

1.3.19. Preparation of N-n-propyl-2,5-diaminoaniline sulfate

Step a) N-n-propyl-2-nitro-5-acetylaminoaniline

Step a) was carried out in the same way as Example 1.3.17. step a) by reacting 2-nitro-5-acetylaminochlorobenzene with n-propylamine.

| | |
|---|---|
| Yield: | 15.5 g(65.3% of the theoretical) of yellow crystals |
| IR: | 3349 cm⁻¹(v CH_{Ar}), 3235, 2964, 2934 cm⁻¹(v CH), 1694 |
| | cm⁻¹(v C═O), 1623 cm⁻¹(v C═C), 1582 cm⁻¹(vₐₛ NO₂), |
| | 1326 cm⁻¹(vₛ NO₂). |
| ¹H-NMR: | 10.30 ppm(1H, s, NHAc); 8.25 ppm(1H, t, NHPr, ³J_{H,H}=5.40 |
| | Hz); 8.02 ppm(H³, d, ³J_{H,H}=9.35 Hz); 7.46 ppm(H⁶, d, ⁴J_{H,H}= |
| | 2.08 Hz); 6.78 ppm(H⁴, dd, ³J_{H,H}=9.39 Hz, ⁴J_{H,H}=2.14 Hz); |
| | 3.22 ppm(2H, m, NC+E,uns H₂); 2.09 ppm(3H, s, C(═O)CH₃); 1.65 |
| | ppm(2H, m, NCH₂C+E,uns H₂CH₃); 0.96 ppm(3H, t, ³J_{H,H}=7.40 Hz, |
| | NCH₂CH₂C+E,uns H₃). |

Step b) N-n-propyl-2-nitro-5-aminoaniline

Step b) was carried out in the same way as Example 1.3.17. step b) by reacting N-n-propyl-2-nitro-5-acetaminoaniline with hydrochloric acid.

| | |
|---|---|
| Yield: | 11.7 g(98.3% of the theoretical) |
| IR: | 3444 cm⁻¹(v CH_{Ar}), 3343, 3232, 2964 cm⁻¹(v CH), 1631 |
| | cm⁻¹(v C═C), 1570 cm⁻¹(vₐₛ NO₂), 1364 cm⁻¹(vₛ NO₂). |
| ¹H-NMR: | 8.38 ppm(1H, t, ³J_{H,H}=4.67 Hz, NH); 7.84 ppm(H³, d, ³J_{H,H}= |
| | 9.36 Hz); 6.59 ppm(2H, s, NH₂); 6.02 ppm(H⁴, dd, ³J_{H,H}= |
| | 9.38 Hz, ⁴J_{H,H}=1.81 Hz); 5.86 ppm(H⁶, s); 3.16 ppm(2H, m, |
| | ³J_{H,H}=7.21 Hz, NC+E,uns H₂); 1.64 ppm(2H, t, ³J_{H,H}=7.37 Hz, |
| | NCH₂₋C+E,uns H₂CH₃); 0.97 ppm(3H, t, ³J_{H,H}=7.37 Hz, |
| | NCH₂CH₂C+E,uns H₃). |

Step c) N-n-propyl-2,5diaminoaniline sulfate

Step c) was carried out in the same way as Example 1.3.17. step c) by catalytic reduction of N-n-propyl-2-nitro-5-aminoaniline.

| | |
|---|---|
| Yield: | 12.1 g(83.5% of the theoretical) |
| Melting point: | >200° C. |
| IR: | 3370 cm⁻¹(v OH), 2932 cm⁻¹(v CH_{Ar}), 1608 cm⁻¹(v |
| | C═C). |
| ¹H-NMR: | 7.8-5.4 ppm(6H, NH₃⁺); 6.78 ppm(H³, d, ³J_{H,H}=8.06 |
| | Hz); 6.71 ppm(H⁴H⁶, m); 2.95 ppm(2H, t, J_{H,H}=7.08 |
| | Hz, NCH₂); 1.62 ppm(2H, q, J_{H,H}=7.16 Hz, NCH₂C+E,uns H₃); |
| | 0.96 ppm(3H, t, ³J_{H,H}=7.34 Hz, NCH₂CH₂C+E,uns H₃). |

1.3.20. Preparation of N-iso-butyl-2,5-diaminoaniline sulfate

Step a) N-iso-butyl-2-nitro-5-acetylaminoaniline

Step a) was carried out in the same way as Example 1.3.17. step a) by reacting 2-nitro-5-acetylaminochlorobenzene with iso-butylamine.

| | |
|---|---|
| Yield: | 14.5 g(57.7% of the theoretical)of yellow crystals |
| IR: | 3347 cm⁻¹(v CH_{Ar}), 3083, 2969, 2936 cm⁻¹(v CH), 1704, |
| | 1680 cm⁻¹(v C═O), 1621 cm⁻¹(v C═C), 1581 cm⁻¹(vₐₛ |
| | NO₂), 1325 cm⁻¹(vₛ NO₂). |
| ¹H-NMR: | 10.31 ppm(1H, s, NHAc); 8.09 ppm(1H, t, NHBu, ³J_{H,H}=7.51 |
| | Hz); 8.08 ppm(H³, d, ³J_{H,H}=9.38 Hz); 7.53 ppm(H⁶, d, |
| | ⁴J_{H,H}=2.01 Hz); 6.78 ppm(H⁴, dd, ³J_{H,H}= 9.38 Hz, |
| | ⁴J_{H,H}=2.09 Hz); 3.53 ppm(1H, qi, C+E,uns H(CH₃)₂); 2.1 |
| | ppm(3H, s, C(═O)CH₃); 1.61 ppm(2H, m, ³J_{H,H}=7.09 |
| | Hz, NC+E,uns H₂(CH₃)₂); 1.24 ppm(3H, d, ³J_{H,H}=6.34 Hz, |
| | syn-CH(C+E,uns H₃)₂); 0.94 ppm(3H, t, ³J_{H,H}=7.42 Hz, |
| | anti-CH(C+E,uns H₃)₂). |

Step b) N-iso-butyl-2-nitro-5-aminoaniline

Step b) was carried out in the same way as Example 1.3.17. step b) by reacting N-iso-butyl-2-nitro-5-acetaminoaniline with hydrochloric acid.

| | |
|---|---|
| Yield: | 11.5 g(55.2% of the theoretical) |
| Melting point: | (red oil) |
| IR: | 3467, 3350, 3234 cm⁻¹(v CH_{Ar}), 2967, 2932, 2876 |
| | cm⁻¹(v CH), 1631 cm⁻¹(v C═C), 1568 cm⁻¹(Vₐₛ NO₂), |
| | 1355 cm⁻¹(vₛ NO₂). |
| ¹H-NMR: | 8.33 ppm(1H, d, ³J_{H,H}=7.56, Hz, NH); 7.82 ppm(H³, d, |
| | ³J_{H,H}=9.46 Hz); 6.00 ppm(H⁴, dd, ³J_{H,H}=9.48 Hz, |
| | ⁴J_{H,H}=2.18 Hz); 5.87 ppm(H⁶, d, ⁴J_{H,H}=2.11 Hz); 3.49 |
| | ppm(1H, m, ³J_{H,H}=6.56 Hz, NCH₂C+E,uns H); 1.59 ppm(2H, |
| | m, ³J_{H,H}=9.21 Hz, NCH₂—C+E,uns H(CH₃)₂); 1.21 ppm(3H, d, |
| | ³J_{H,H}=6.33 Hz, anti-NCH₂C+E,uns H—(CH₃)₂); 0.93 ppm(3H, t, |
| | ³J_{H,H}=7.44 Hz, syn-NCH₂C+E,uns H(CH₃)₂). |

Step c) 3-iso-butylamino-4-aminoaniline sulfate

Step c) is carried out in the same way as Example 1.3.17. step c) by catalytic reduction of N-iso-butyl-2-nitro-5-aminoaniline.

| | |
|---|---|
| Yield: | 8 g(55.5% of the theoretical) |
| Melting point: | >200° C. |
| IR: | 3393 cm⁻¹(v OH), 2968 cm⁻¹(v CH_{Ar}), 1608 cm⁻¹(v |
| | C═C). |
| ¹H-NMR: | 7.8-7.0 ppm(6H, NH₃⁺); 6.69 ppm(H³, d, ³J_{H,H}=8.15 |
| | Hz); 6.19 ppm(H⁶, s); 6.13 ppm(H⁴, d, ³J_{H,H}=8.10 Hz); |
| | 3.27 ppm(1H, m, NHCH₂C+E,uns H(CH₃)₂); 1.59 ppm(1H, m, |
| | ³J_{H,H}=6.77 Hz, anti-NC+E,uns H₂); 1.46ppm(1H, m, J_{H,H}= |
| | 6.91 Hz, syn-NC+E,uns H₂); 1.14 ppm(3H, m, ³J_{H,H}=6.22 Hz, |
| | anti-NCH₂CH(C+E,uns H₃)); 0.93 ppm(3H, m, J_{H,H}=7.33 Hz, |
| | syn-NC+E,uns H₂). |

1.3.21. Preparation of N,N-dimethyl-2,5-diaminosulfate

Step a) N, N-dimethyl-2-nitro-5-acetylaminoaniline

Step a) was carried out in the same way as Example 1.3.17. step a) by reacting 2-nitro-5-acetylaminochlorobenzene with dimethylamine.

| | |
|---|---|
| Yield: | 16.8 g(75.5% of the theoretical)of yellow crystals |
| IR: | 3552, 3363 cm⁻¹(v CH_{Ar}), 2928, 2801 cm⁻¹(v CH), 1678 |
| | cm⁻¹(v C═O), 1613 cm⁻¹(v C═C), 1554 cm⁻¹(vₐₛ NO₂), |
| | 1337 cm⁻¹(vₛ NO₂). |
| ¹H-NMR: | 10.2 ppm(1H, s, NHAc); 7.8 ppm(H³, d, ³J_{H,H}=9.02 Hz); 7.48 |
| | ppm(H⁶, d, ⁴J_{H,H}=1.83 Hz); 7.05 ppm(H⁴, dd, ³J_{H,H}=9.03 Hz, |
| | ⁴J_{H,H}=1.98 Hz); 2.08 ppm(3H, s, C(═O)CH₃); 2.78 ppm(6H, |
| | s, N(CH₃)₂). |

Step b) N,N-dimethyl-2-nitro-5-aminoaniline

Step b) was carried out in the same way as Example 1.3.17. step b) by reacting N,N-dimethyl-2-nitro-5-acetaminoaniline with hydrochloric acid.

| | |
|---|---|
| Yield: | 18.9 g(100% of the theoretical)of yellow crystals |
| IR: | 3449, 3337 cm⁻¹(v CH_{Ar}), 2971, 2872 cm⁻¹(v CH), 1619 |
| | cm⁻¹(v C═C), 1562 cm⁻¹(Vₐₛ NO₂), 1320 cm⁻¹(vₛ NO₂). |
| ¹H-NMR: | 7.83 ppm(H³, d, ³J_{H,H}=9.70 Hz); 7.29 ppm(2H, s, NH₂); 6.38 |
| | ppm(H⁴, dd, ³J_{H,H}=9.76 Hz, ⁴J_{H,H}=2.60 Hz); 6.23 ppm(H⁶, d, |
| | ⁴J_{H,H}=2.60 Hz); 3.71 ppm(3H, t, ³J_{H,H}=4.88 Hz, syn-NCH₃); |
| | 3.27 ppm(3H, t, ³J_{H,H}=4.90 Hz, anti-NCH₃). |

Step c) N,N-dimethyl-2,5-diaminoaniline sulfate

Step c) was carried out in the same way as Example 1.3.17. step c) by catalytic reduction of N,N-dimethyl-2-nitro-5-aminoaniline.

| | |
|---|---|
| Yield: | 19.6 g(81.1% of the theoretical) |
| Melting point: | >200° C. |
| IR: | 3368, 3224 cm⁻¹(v OH), 2851 cm⁻¹(v CH_{Ar}), 1630 cm⁻¹ |
| | (v C═C). |
| ¹H-NMR: | 8.0-7.0 ppm(6H, NH₃⁺); 7.0 ppm(H³, d, ³J_{H,H}=8.65 Hz); |
| | 6.43 ppm(H⁶, d, ³J_{H,H}=2.46 Hz); 6.31 ppm(H⁴, dd, |
| | ³J_{H,H}=8.73 Hz, ⁴J_{H,H}=2.46 Hz); 3.72 ppm(3H, t, |
| | ³J_{H,H}=4.69 Hz, syn-NCH₃); 3.03 ppm(3H, t, ³J_{H,H}=4.71 |
| | Hz, anti-NCH₃). |

1.3.22. Preparation of N,N-diethyl-2,5-diaminoaniline sulfate

Step a) N,N-diethyl-2-nitro-5-acetylaminoaniline

Step a) was carried out in the same way as Example 1.3.17. step a) by reacting 2-nitro-5-acetylaminochlorobenzene with diethylamine.

| | |
|---|---|
| Yield: | 20.1 g(79.9% of the theoretical) of yellow crystals |
| IR: | 3437, 3294 cm⁻¹(v CH_{Ar}), 2979, 2934 cm⁻¹(v CH), 1671 |
| | cm⁻¹(v C═O), 1613 cm⁻¹(v C═C), 1558 cm⁻¹(vₐₛ NO₂), |
| | 1347 cm⁻¹(vₛ NO₂). |
| ¹H-NMR: | 10.43 ppm(1H, s, NHAc); 7.92 ppm(H³, d, ³J_{H,H}=9.24 Hz); |
| | 7.75 ppm(H⁶, d, ⁴J_{H,H}=2.06 Hz); 7.35 ppm(H⁴, dd, ³J_{H,H}= |
| | 8.97 Hz, ⁴J_{H,H}=2.06 Hz); 3.26 ppm(4H, q, ³J_{H,H}=7.04 Hz, |
| | N(C+E,uns H₂CH₃)₂); 2.26 ppm(3H, s, C(═O)CH₃); 1.20 ppm(6H, t, |
| | ³J_{H,H}=7.04 Hz, N(CH₂C+E,uns H₃)₂). |

Step b) N,N-diethyl-2-nitro-5-aminoaniline

Step b) was carried out in the same way as Example 1.3.17. step b) by reacting N,N-diethyl-2-nitro-5-acetaminoaniline with hydrochloric acid.

| | |
|---|---|
| Yield: | 12.6 g(60.2% of the theoretical) |
| Melting point: | (red oil) |
| IR: | 3469, 3367, 3233 cm⁻¹(v CH_{Ar}), 2974, 2923, 2872 |
| | cm⁻¹(v CH), 1631 cm⁻¹(v C═C), 1567 cm⁻¹(vₐₛ NO₂), |
| | 1344 cm⁻¹(vₛ NO₂). |
| ¹H-NMR: | 7.91 ppm(H³, d, ³J_{H,H}=8.99 Hz); 6.45 ppm(2H, s, |
| | NH₂); 6.41 ppm(H⁶, d, ⁴J_{H,H}=2.23 Hz); 6.32 ppm(H⁴, |
| | dd, ³J_{H,H}=9.08 Hz, ⁴J_{H,H}=2.23 Hz); 3.23 ppm(3H, t, |
| | ³J_{H,H} =7.04 Hz, N(C+E,uns H₂—CH₃)₂); 1.21 ppm(3H, t, |
| | ³J_{H,H}=7.03 Hz, N(CH₂C+E,uns H₃)₂). |

Step c) N,N-diethyl-2,5-diaminoaniline sulfate

Step c) was carried out in the same way as Example 1.3.17. step c) by catalytic reduction of N,N-diethyl-2-nitro-5-aminoaniline.

| | |
|---|---|
| Yield: | 10.1 g(64.3% of the theoretical) |
| Melting point: | >200° C. |
| IR: | 3379 cm⁻¹(v OH), 3233 cm⁻¹(v CH_{Ar}), 2901 cm⁻¹(v |
| | CH_{Al}), 1630 cm⁻¹(v C═C). |
| ¹H-NMR: | 8.4-7.0 ppm(6H, NH₃⁺); 6.84 ppm(H³, d, ³J_{H,H}=8.51 |
| | Hz); 6.81 ppm(H⁶, d, ³J_{H,H}=2.41 Hz); 6.7 ppm(H⁴, dd, |
| | ³J_{H,H}=8.41 Hz, ⁴J_{H,H}=2.13 Hz); 2.91 ppm(3H, t, |
| | ³J_{H,H}=7.04 Hz, N(C+E,uns H₂—CH₃)₂); 0.93 ppm(3H, t, |
| | ³J_{H,H}=7.05 Hz, N(CH₂C+E,uns H₃)₂). |

1.3.23. Preparation of N-(2, 5-diaminophenyl)pyrrolidine sulfate

Step a) N-(5acetylamino-2-nitrophenyl)pyrrolidine

Step a) was carried out in the same way as Example 1.3.17. step a) by reacting 2-nitro-5-acetylaminochlorobenzene with pyrrolidine.

| | |
|---|---|
| Yield: | 24.3 g(97.5% of the theoretical) of yellow crystals |
| IR: | 3262 cm⁻¹(v CH_{Ar}), 2969, 2873 cm⁻¹(v CH), 1667 cm⁻¹(v |
| | C═O), 1614 cm⁻¹(v C═C), 1548 cm⁻¹(vₐₛ NO₂), 1358 |
| | cm⁻¹(vₛ NO₂). |
| ¹H-NMR: | 10.38 ppm(1H, s, NHAc); 7.91 ppm(H³, d, ³J_{H,H}=9.05 Hz); |
| | 7.61 ppm(H⁶, d, ⁴J_{H,H}=1.95 Hz); 7.11 ppm(H⁴, dd, ³J_{H,H}= |
| | 9.06 Hz, ⁴J_{H,H}=2.01 Hz); 3.27 ppm(4H, t, ³J_{H,H}=6.25 Hz, |
| | N(CH₂)₂); 2.25 ppm(3H, s, C(═O)CH₃); 2.09 ppm(4H, t, |
| | ³J_{H,H}=6.25 Hz, N(CH₂C+E,uns H₂)₂). |

Step b) N-(5amino-2-nitrophenyl)pyrrolidine

Step b) was carried out in the same way as Example 1.3.17. step b) by reacting N-(5-acetylamino-2-nitrophenyl)pyrrolidine with hydrochloric acid.

| | |
|---|---|
| Yield: | 18.3 g(98.1% of the theoretical) |
| Melting point: | (red oil) |
| IR: | 3468, 3366, 3231 cm⁻¹(v CH_{Ar}), 2970, 2874 cm⁻¹(v |
| | CH), 1608 cm⁻¹(v C═C), 1560 cm⁻¹(vₐₛ NO₂), 1360 |
| | cm⁻¹(vₛ NO₂). |
| ¹H-NMR: | 7.81 ppm(H³, d, ³J_{H,H}=9.35 Hz); 6.32 ppm(2H, s, NH₂); |
| | 6.18 ppm(H⁴H⁶, m); 3.25 ppm(4H, t, ³J_{H,H}=6.34 Hz, |
| | N(C+E,uns H₂CH₂)₂); 2.05 ppm(4H, t, ³J_{H,H}=6.35 Hz, |
| | N(CH₂C+E,uns H₂)₂). |

Step c) N-(2,5-diaminophenyl)pyrrolidine sulfate

Step c) was carried out in the same way as Example 1.3.17. step c) by catalytic reduction of N-(5-amino-2-nitrophenyl)pyrrolidine.

| | |
|---|---|
| Yield: | 13.5 g(59.5% of the theoretical) |
| Melting point: | >200° C. |
| IR: | 3382 cm⁻¹(v OH), 3231 cm⁻¹(v CH_{Ar}), 2882 cm⁻¹(v |
| | CH_{Al}), 1625 cm⁻¹(v C═C). |
| ¹H-NMR: | 8.0-6.4 ppm(6H, NH₃⁺); 6.82 ppm(H³, d, ³J_{H,H}=8.35 |
| | Hz); 6.57 ppm(H⁶, d, ³J_{H,H}=2.06 Hz); 6.45 ppm(H⁴, dd, |
| | ³J_{H,H}=8.33 Hz, ⁴J_{H,H}=2.06 Hz); 3.03 ppm(4H, t, |
| | ³J_{H,H}=5.86 Hz, N(C+E,uns H₂CH₂)₂); 1.88 ppm(4H, t, |
| | ³J_{H,H}=5.98 Hz, N(CH₂C+E,uns H₂)₂). |

1.3.24. Preparation of N-(2,5-diaminophenyl)piperidine sulfate

Step a) N-(5-acetylamino-2-nitrophenyl)piperidine

Step a) was carried out in the same way as Example 1.3.17. step a) by reacting 2-nitro-5-acetylaminochlorobenzene with piperidine.

| | |
|---|---|
| Yield: | 22.1 g(84.1% of the theoretical) of yellow crystals |

Step b) N-(5-amino-2-nitrophenyl)piperidine

Step b) was carried out in the same way as Example 1.3.17. step b) by reacting N-(5-acetylamino-2-nitrophenyl)piperidine with hydrochloric acid.

| | |
|---|---|
| Yield: | 17.8 g(81.5% of the theoretical) |
| Melting point: | (red oil) |
| IR: | 3449, 3358, 3245 cm⁻¹(v CH_{Ar}), 2990, 2938 cm⁻¹(v |
| | CH), 1604 cm⁻¹(v C═C), 1563 cm⁻¹(vₐₛ NO₂), 1341 |
| | cm⁻¹(vₛ NO₂). |
| ¹H-NMR: | 7.83 ppm(H³, d, ³J_{H,H}=8.91 Hz); 6.40 ppm(2H, s, NH₂); |
| | 6.19 ppm(H⁴, dd, ³J_{H,H}=4.36 Hz, ⁴J_{H,H}=2.19 Hz); 6.15 |
| | ppm(H⁶, d, ⁴J_{H,H}=2.29 Hz); 2.88 ppm(4H, ³J_{H,H}=5.37 |
| | Hz, N(C+E,uns H₂CH₂)₂—CH₂); 1.63 ppm(4H, m, |
| | N(CH₂C+E,uns H₂)₂CH₂); 1.54 ppm(2H, m, |
| | N(CH₂CH₂)₂C+E,uns H₂). |

Step c) N-(2,5-diaminophenyl)piperidine sulfate

Step c) was carried out in the same way as Example 1.3.17. step c) by catalytic reduction of N-(5-amino-2-nitrophenyl)piperidine.

| | |
|---|---|
| Yield: | 13.3 g(57.5% of the theoretical) |
| Melting point: | >200° C. |

1.3.25. Preparation of N-(2,5-diaminophenyl)azepan sulfate

Step a) N-(5-acetylamino-2-nitrophenyl)azepan

Step a) was carried out in the same way as Example 1.3.17. step a) by reacting 2-nitro-5-acetylaminochlorobenzene with azepan.

| | |
|---|---|
| Yield: | 25.3 g(91.3% of the theoretical) of yellow crystals |
| IR: | 3331, 3285 cm⁻¹(v CH_{Ar}), 2934, 2856 cm⁻¹(v CH), 1700, |
| | 1681 cm⁻¹(v C═O), 1613 cm⁻¹(v C═C), 1549 cm⁻¹(vₐₛ |
| | NO₂), 1338 cm⁻¹(vₛ NO₂). |
| ¹H-NMR: | 10.2 ppm(1H, s, NHAc); 7.70 ppm(H³, d, ³J_{H,H}=9.02 Hz); |
| | 7.62 ppm(H⁶, d, ⁴J_{H,H}=1.85 Hz); 6.97 ppm(H⁴, dd, |
| | ³J_{H,H}=8.98 Hz, ⁴J_{H,H}=1.83 Hz); 3.20 ppm(4H, t, ³J_{H,H}=5.57 |
| | Hz, N(CH₂)₂); 2.08 ppm(3H, s, C(═O)CH₃); 1.76 ppm(4H, |
| | s, N(CH₂C+E,uns H₂)₂); 1.52 ppm(4H, s, N(CH₂CH₂C+E,uns H₂)₂). |

Step b) N-(5-amino-2-nitrophenyl)azepan

Step b) was carried out in the same way as Example 1.3.17. step b) by reacting N-(5-acetylamino-2-nitrophenyl)azepan with hydrochloric acid.

| | |
|---|---|
| Yield: | 19.8 g(95.6% of the theoretical) of yellow crystals |
| IR: | 3460, 3353 cm⁻¹(v CH_{Ar}), 3230, 2926, 2855 cm⁻¹(v CH), |
| | 1607 cm⁻¹(v C═C), 1550 cm⁻¹(vₐₛ NO₂), 1362 cm⁻¹(vₛ |
| | NO₂). |
| ¹H-NMR: | 7.65 ppm(H³, d, ³J_{H,H}=9.08 Hz); 6.19 ppm(H⁶, d, ⁴J_{H,H}=1.95 |
| | Hz); 6.16 ppm(2H, s, NH₂); 6.05 ppm(H⁴, dd, ³J_{H,H}=9.02 Hz, |
| | ⁴J_{H,H}=1.85 Hz); 3.18 ppm(4H, t, ³J_{H,H}=5.48 Hz, N(CH₂)₂); |
| | 1.72 ppm(4H, s, N(CH₂C+E,uns H₂)₂); 1.53 ppm(4H, s, |
| | N(CH₂CH₂—C+E,uns H₂)₂). |

Step c) N-(2,5-diaminophenyl)azepan sulfate

Step c) was carried out in the same way as Example 1.3.17. step c) by catalytic reduction of N-(5-amino-2-nitrophenyl)azepan. P Melting point: >200° C.

| | |
|---|---|
| IR: | 3368 cm⁻¹(v OH), 3214 cm⁻¹(v CH_{Ar}), 2926 cm⁻¹(v CH_{Al}), |
| | 1618 cm⁻¹(v C═C). |
| ¹H-NMR: | 8.4-6.4 ppm(6H, NH₃⁺); 6.82 ppm(H³H⁶, m); 6.65 ppm(H⁴, |
| | dd, ³J_{H,H}=8.41 Hz, ⁴J_{H,H}=2.27 Hz); 2.95 ppm(4H, t, ³J_{H,H}= |
| | 5.41 Hz, N(C+E,uns H₂CH₂)₂); 1.70 ppm(8H, m, N(CH₂C+E,uns H₂C+E,uns H₂)₂). |

1.3.26. Preparation of N-(2,5-diaminophenyl)morpholine sulfate

Step a) N-(5-acetylamino-2-nitrophenyl)morpholine

Step a) was carried out in the same way as Example 1.3.17. step a) by reacting 2-nitro-5-acetylaminochlorobenzene with morpholine.

| | |
|---|---|
| Yield: | 25.6 g(96.5% of the theoretical) of yellow crystals |
| IR: | 3291 cm⁻¹(v CH_{Ar}), 2968, 2836 cm⁻¹(v CH), 1698 cm⁻¹(v |
| | C═O), 1612 cm⁻¹(v C═C), 1550 cm⁻¹(vₐₛ NO₂), 1331 |
| | cm⁻¹(vₛ NO₂). |
| ¹H-NMR: | 10.54 ppm(1H, s, NHAc); 8.1 ppm(H³, d, ³J_{H,H}=9.02 Hz); |
| | 7.71 ppm(H⁶, d, ⁴J_{H,H}=2.08 Hz); 7.48 ppm(H⁴, dd, |
| | ³J_{H,H}=8.97 Hz, ⁴J_{H,H}=2.08 Hz); 3.9 ppm(4H, t, ³J_{H,H}=4.54 |
| | Hz, N,N(CH₂—C+E,uns H₂O)₂); 3.13 ppm(4H, t, ³J_{H,H}=4.56 Hz, |
| | N(C+E,uns H₂CH₂O)₂); 2.27 ppm(3H, s, C(═O)CH₃). |

Step b) N-(5-amino-2-nitrophenyl)morpholine

Step b) was carried out in the same way as Example 1.3.17. step b) by reacting N-(5-acetylamino-2-nitrophenyl)morpholine with hydrochloric acid.

| | |
|---|---|
| Yield: | 19.2 g(95.6% of the theoretical) of yellow crystals |
| Melting point: | (red oil) |
| IR: | 3466, 3320, 3220 cm⁻¹ (v CH_{Ar}), 2970, 2867 cm⁻¹ (v |
| | CH), 1606 cm⁻¹ (v C═C), 1572 cm⁻¹ |
| | (Vₐₛ NO₂), 1344 cm⁻¹ (vₛ NO₂). |
| ¹H-NMR: | 7.88 ppm (H³, d, ³J_{H,H} = 8.63 Hz); |
| | 6.51 ppm (2H, s, NH₂); 6.22 ppm (H⁴H⁶, m); 3.73 ppm |
| | (4H, t, ³J_{H,H} = 4.44 Hz, N(CH₂C+E,uns H₂O)₂); 2.92 ppm |
| | (4H, t, ³J_{H,H} = 4.47 Hz, N(CH₂C+E,uns H₂O)₂). |

Step c) N-(2,5-diaminophenyl)morpholine sulfate

Step c) was carried out in the same way as Example 1.3.17. step c) by catalytic reduction of N-(5-amino-2-nitrophenyl)morpholine.

| | |
|---|---|
| Yield: | 22.3 g (95.7% of the theoretical) |
| Melting point: | >200° C. |
| IR: | 3411 cm⁻¹ (v CH_{Ar}), 2873 cm⁻¹ (v CH_{Al}), 1613 |
| | cm⁻¹ (v C═C). |
| ¹H-NMR: | 8.4-6.6 ppm (6H, NH₃⁺); 6.83 ppm (H³, d, ³J_{H,H} = 8.44 |
| | Hz); 6.81 ppm (H⁶, d, ³J_{H,H} = 2.02 Hz); 6.72 ppm (H⁴, |
| | dd, ³J_{H,H} = 8.34 Hz, ⁴J_{H,H} = 1.84 Hz); 3.76 ppm (4H, t, |
| | ³J_{H,H} = 4.33 Hz, N(CH₂C+E,uns H₂O)₂); |
| | 2.78 ppm (4H, t, ³J_{H,H} = 4.31 Hz, N(CH₂C+E,uns H₂O)₂). |

1.3.27. Preparation of N-(2,5-diaminophenyl)piperazine sulfate

Step a) N-(5-acetylamino-2-nitrophenyl)piperazine

Step a) was carried out in the same way as Example 1.3.17. step a) by reacting 2-nitro-5-acetylaminochlorobenzene with piperazine.

| | |
|---|---|
| Yield: | 21.5 g (81% of the theoretical) of yellow crystals |
| IR: | 3435 cm⁻¹ (v CH_{Ar}), 3039, 2920, 2853 cm⁻¹ (v CH), |
| | 1695 cm⁻¹ (v C═O), 1615 cm⁻¹ (v C═C), 1558 cm⁻¹ (vₐₛ |
| | NO₂), 1366 cm⁻¹ (vₛ NO₂). |
| ¹H-NMR; | 10.32 ppm (1H, s, NHAc); 7.86 ppm (H³, d, ³J_{H,H} 9.01 Hz); |
| | 7.51 ppm (H⁶, d, ⁴J_{H,H} = 1.93 Hz); 7.25 ppm (H⁴, dd, ³J_{H,H} = |
| | 8.99 Hz, ⁴J_{H,H} = 1.97 Hz); 2.86 ppm (4H, t, ³J_{H,H} = 5.65 Hz, |
| | N(CH₂C+E,uns H₂NH)₂); 2.82 ppm (4H, t, ³J_{H,H} = 5.67 Hz, |
| | N(C+E,uns H₂CH₂NH)₂); 2.09 ppm (3H, s, C(═O)CH₃). |

Step b) N-(5-amino-2-nitrophenyl)piperazine

Step b) was carried out in the same way as Example 1.3.17. step b) by reaction of N-(5-acetylamino-2-nitrophenyl)piperazine with hydrochloric acid.

| | |
|---|---|
| Yield: | 14.6 g (93.8% of the theoretical) of yellow crystals |
| IR: | 3411, 3317, 3217 cm⁻¹ (v CH_{Ar}), 2962, 2837 cm⁻¹ (v CH), |
| | 1608 cm⁻¹ (v C═C), 1567 cm⁻¹ (vₐₛ NO₂), |
| | 1349 cm⁻¹ (vₛ NO₂). |
| ¹H-NMR: | 7.88 ppm (H³, d, ³J_{H,H} = 9.66 Hz); 6.59 ppm (2H, |
| | s, NH₂); 6.26 ppm (H⁴, dd, ³J_{H,H} = 9.56 Hz, ⁴J_{H,H} = |
| | 2.18 Hz); 6.25 ppm (H⁶, d, ⁴J_{H,H} = 2.07 Hz); 5.2 ppm (1H, |
| | s, NH); 3.02 ppm (8H, s, N(C+E,uns H₂CH₂NH)₂). |

Step c) N-(2,5-diaminophenyl)piperazine sulfate

Step c) was carried out in the same way as Example 1.3.17. step c) by catalytic reduction of N-(5-amino-2-nitrophenyl)piperazine.

| | |
|---|---|
| Yield: | 13.2 g (75.8% of the theoretical) |
| Melting point: | >200° C. |
| IR: | 3436 cm⁻¹ (v CH_{Ar}), 2848 cm⁻¹ (v CH_{Al}), 1616 |
| | cm⁻¹ (v C═C). |
| ¹H-NMR: | 8.8-6.8 ppm (6H, NH₃⁺); 6.66 ppm (H³, s); 6.58 ppm |
| | (H⁴H⁶, d); 3.28 ppm (4H, s, N(CH₂C+E,uns H₂NH⁺₂)₂); |
| | 2.97 ppm (4H, s, N(C+E,uns H₂CH₂NH⁺₂)₂); 2.51 ppm (2H, s, |
| | N(CH₂CH₂N+E,uns H₂⁺)₂). |

1.3.28. Preparation of N,N-dimethyl-3,4-diaminoaniline sulfate

Step a) N,N-dimethyl-4-nitro-3-acetaminoaniline

100 ml of 1,2-dimethoxyethane, 21.5 g (0.1 mole) of 4-nitro-3-acetaminochlorobenzene, 20.7 g (0.15 mole) of potassium carbonate and 16.9 g (0.15 mole) of 40% dimethylamine solution were introduced into an autoclave. The mixture was then heated with stirring for 8 hours to 120° C. The hot reaction mixture was then filtered off from the inorganic salts and the solvent was distilled off in vacuo. The yellow powder precipitated was recrystallized from ethanol.

| | |
|---|---|
| Yield: | 21.3 g (95.4% of the theoretical) |
| IR: | 3313 cm⁻¹ (v CH_{Ar}), 3313, 3143, 2927 cm⁻¹ (v CH), |
| | 1702 cm⁻¹ (v C═O), 1622 cm⁻¹ (v C═C), 1575, 1548 cm⁻¹ |
| | (vₐₛ NO₂), 1344, 1310 cm⁻¹ (vₛ NO₂). |
| ¹H-NMR: | 10.59 ppm (1H, s, NHAc); 8.02 ppm (H⁵, d, ³J_{H,H} = 9.70 Hz); |
| | 7.63 ppm (H², d, ⁴J_{H,H} = 2.71 Hz); 6.54 ppm (H⁶, dd, ³J_{H,H} = |
| | 9.57 Hz, ⁴J_{H,H} = 2.55 Hz); 3.07 ppm (6H, s, N(C+E,uns H₃)₂); 2.17 |
| | ppm (3H, s, C(═O)CH₃). |

Step b) N,N-dimethyl-2-nitro-5-aminoani/ine

20.3 g (0.091 mole) of N,N-dimethyl-4-nitro-3-acetaminoaniline (from step a) were introduced into a mixture of 75 ml of water and 21 ml of methanol and the whole was heated under reflux for 1 hour with 20.7 ml of concentrated hydrochloric acid. On completion of the reaction, the mixture was adjusted to pH 7 with ammonia and the product was separated by stirring, filtered off under suction and washed with water.

| | |
|---|---|
| Yield: | 15.9 g (96.4% of the theoretical) |
| IR: | 3468, 3348 cm⁻¹ (v CH_{Ar}), 2922 cm⁻¹ (v CH), 1619 cm⁻¹ |
| | (v C═C), 1557 cm⁻¹ (vₐₛ NO₂), 1312 cm⁻¹ (vₛ NO₂). |
| ¹H-NMR: | 7.82 ppm (H⁵, d, ³J_{H,H} = 9.75 Hz); 7.25 ppm (2H, s, NH₂); |
| | 6.20 ppm (H⁶, dd, ³J_{H,H} = 9.81 Hz, ⁴J_{H,H} = 2.68 Hz); 5.96 |
| | ppm (H², d, ⁴J_{H,H} = 2.67 Hz); 3.00 ppm (6H, s, N(C+E,uns H₃)₂). |

Step c) N,N-dimethyl-3,4-diaminoaniline sulfate

14.9 g (0.082 mole) of N,N-dimethyl-2-nitro-5-aminoaniline (from step b) were introduced into a 0.3 liter autoclave with 1 g of palladium on active carbon 10% (Degussa) in 180 ml of methanol. After the autoclave had been closed and blanketed with nitrogen, hydrogenation was carried out under a pressure of 3 bar and at a temperature of 35 to 40° C. until no more hydrogen was taken up. 1.3 g of active carbon was added under nitrogen to the warm solution and the catalyst was filtered off. 8.4 g of 80% sulfuric acid were added dropwise to the solution while cooling with ice at 5° C. The product precipitated was filtered off under suction, washed with methanol and dried.

| | |
|---|---|
| Yield: | 10.1 g (49.4% of the theoretical) |
| Melting point: | >200° C. |
| IR: | 3300 cm⁻¹ (v OH), 3222, 2856 cm⁻¹(v CH_{Ar}), 1641 cm⁻¹ |
| | (v C═C). |
| ¹H-NMR: | 7.6-6.4 ppm (6H, NH₃⁺); 6.94 ppm (H⁵, d, ³J_{H,H} = |
| | 8.68 Hz); 6.30 ppm (H², d, ⁴J_{H,H} = 2.47 Hz), 6.18 ppm |
| | (H⁶, dd, ³J_{H,H} = 8.70 Hz, ⁴J_{H,H} = 2.47 Hz); 2.88 ppm |
| | (6H, s, N(C+E,uns H₃)₂). |

1.3.29. Preparation of N-(3,4-diaminophenyl)-morpholine sulfate

Step a) N-(4-nitro-3-acetaminophenyl)-morpholine

Step a) was carried out in the same way as Example 1.3.28. step a) by reacting 4-nitro-3-acetaminochlorobenzene with morpholine.

| | |
|---|---|
| Yield: | 24.1 g (90.8% of the theoretical) of yellow crystals |
| IR: | 3438 cm⁻¹ (v CH_{Ar}), 3312, 3140, 2973, 2855 cm⁻¹ (v CH), |
| | 1698 cm⁻¹ (v C═O), 1617 cm⁻¹ (v C═C), 1580 cm⁻¹ |
| | (vₐₛ NO₂), 1312 cm⁻¹ (vₛ NO₂). |
| ¹H-NMR: | 10.43 ppm (1H, s, NHAc); 7.98 ppm (H⁵, d, ³J_{H,H} = 9.55 Hz); |
| | 7.70 ppm (H², d, ⁴J_{H,H} = 2.69 Hz); 6.78 ppm (H⁶, dd, |
| | ³J_{H,H} = 9.59 Hz, ⁴J_{H,H} = 2.72 Hz); 3.73 ppm (4H, t, ³J_{H,H} = |
| | 4.85 Hz, N(CH₂C+E,uns H₂)₂O); 3.35 ppm (4H, t, ³J_{H,H} = 4.91 Hz, |
| | N(C+E,uns H₂CH₂)₂O); 2.15 ppm (3H, s, C(═O)C+E,uns H₃). |

Step b) N-(4-nitro-3-aminophenyl)-morpholine

Step b) was carried out in the same way as Example 1.3.28. step b) by reacting N-(4-nitro-3-acetaminophenyl)-morpholine with hydrochloric acid.

Yield: 15.3 g (84.4% of the theoretical)

Step c) N-(3,4-diaminophenyl)-morpholine sulfate

Step c) was carried out in the same way as Example 1.3.28. step c) by catalytic reduction of N-(4-nitro-3-aminophenyl)-morpholine.

| | |
|---|---|
| Yield: | 15.4 g(85.8% of the theoretical) |
| Melting point: | >200° C. |

2. Coloring 2.1 Colorants:

| | |
|---|---|
| Creme-based C1 | |
| Sodium lauryl sulfate (70%) | 2.5 g |
| Oleic acid | 1.0 g |
| Sodium sulfite, anhydrous | 0.6 g |
| Cetostearyl alcohol | 12.0 g |
| Myristyl alcohol | 6.0 g |
| Propylene glycol | 1.0 g |
| Ammonia, 25% | 10.0 g |
| Oxidation dye precursors | x.x g |
| Water | to 100 g |
| Creme-based C2 | |
| Oleic acid | 1.2 g |
| Sodium dithionite | 0.5 g |
| Lauryl alcohol diglycol ether sulfate, | 6.2 g |
| sodium salt (28% solution) | |
| Cetostearyl alcohol | 18.0 g |
| Ammonia, 25% | 7.5 g |
| Oxidation dye precursors | x.x g |
| Water | to 100 g |
| Gel-based G1 | |
| Oleic acid | 12.0 g |
| Isopropanol | 12.0 g |
| Nonoxynol-4 | 5.0 g |
| Ammonia, 25% | 10.0 g |
| Sodium sulfite, anhydrous | 0.5 g |
| Oxidation dye precursors | x.x g |
| Water | 10 100 g |

2.2 Oxidation dye precursors corresponding to formula (I)

(I-1) N-(2,4-diaminophenyl)-ethanolamine sulfate

(I-2) 2,4-diamino-N,N-diethyl aniline sulfate

(I-3) N-(2,4-diaminophenyl)-pyrrolidine sulfate

(I-4) N,N-dimethyl-2,4-diaminoaniline sulfate

(I-5) N-(2,4-diaminophenyl)morpholine sulfate

(I-6) N-[4-(2-hydroxyethylamino)-2-aminophenyl]piperidine sulfate

(I-7) N-{2-amino-4-[di-(2-hydroxyethyl)amino]phenyl}pyrrolidine sulfate

(I-8) N-[N,N-diethylaminoethyl]-2-amino-4-(3-hydroxypropylamino)aniline sulfate

(I-9) N-[2-amino-4-(3-hydroxypropylamino)phenyl]azepan sulfate

(I-10) N,N-dimethyl-2,5-diaminoaniline sulfate

(I-11) N-(2,5-diaminophenyl)morpholine sulfate

(I-12) N-(2,5-diaminophenyl)pyrrolidine sulfate

(I-13) N,N-diethyl-2,5-diaminoaniline

(I-14) N-(2,5-diaminophenyl)piperazine

(I-15) N-methyl-2,5-diaminoaniline sulfate

(I-16) N,N-dimethyl-3,4-diaminoaniline sulfate

Primary intermediates

(P-1) p-aminophenol

(P-2) 2-(2′-hydroxyethyl)-p-phenylenediamine sulfate

(P-3) p-phenylenediamine dihydrochloride

(P-4) 2,5-diaminotoluene sulfate

(P-5) 4-amino-m-cresol

Secondary intermediates

(S-1) resorcinol

(S-2) m-aminophenol

(S-3) 4-amino-2-hydroxytoluene

(S4) 2-amino4-hydroxyethylaminoanisole sulfate

2.3. Procedure

50 g of the colorant were mixed just before use with 50 g of H₂O₂ solution (6% in water) and the resulting mixture was applied by brush to 100% grey hair (4 g of colorant per g of hair). After a contact time of 30 minutes at room temperature, the creme-based colorant was rinsed off and the hair was dried. In the case of the gel base, the hair was shampooed and dried after the colorant had been rinsed off.

2.4. Results

The coloring results are set out in the following Table:

| Base | Oxidation dye precursors | Color |
|---|---|---|
| C1 | 2.65 g I-1 + 1.09 g P-1 | Uniform light brown-red |
| C1 | 2.65 g I-1 + 1.52 g P-2 | Uniform mid-brown |
| C1 | 2.65 g I-1 + 1.08 g P-4 | Uniform dark brown |
| C1 | 2.65 g I-1 + 1.23 g P-5 | Uniform light-brown-red |
| C1 | 2.65 g I-4 + 1.10 g S-1 | Uniform olive-orange |
| C1 | 2.65 g I-4 + 1.09 g S-2 | Uniform green-blue |
| C1 | 2.65 g I-4 + 1.23 g S-3 | Uniform azure blue |
| C1 | 2.65 g I-4 + 2.80 g S-4 | Uniform marine blue |
| G1 | 2.66 g I-2 + 1.10 g S-1 | Brown violet |
| G1 | 2.66 g I-2 + 1.09 g P-1 | Light golden blond |
| G1 | 2.66 g I-2 + 1.52 g P-1 | Light ash blond |
| G1 | 2.66 g I-2 + 1.08 g P-3 | Light ash violet |
| G1 | 2.66 g I-2 + 1.22 g P-4 | Light ash blond |
| G1 | 2.66 g I-2 + 1.23 g P-5 | Light golden blond |
| C1 | 2.75 g I-3 + 1.09 g P-1 | Medium golden blond |
| C1 | 2.49 g I-10 + 1.10 g S-1 | Uniform olive orange |
| C1 | 2.49 g I-10 + 1.09 g S-2 | Uniform green-blue |
| C1 | 2.49 g I-10 + 1.23 g S-3 | Uniform azure blue |
| C1 | 2.49 g I-10 + 2.80 g S-4 | Uniform marine blue |
| G1 | 2.91 g I-11 + 1.10 g S-1 | Brown-violet |
| G1 | 2.91 g I-11 + 1.09 g S-2 | Blue-grey |
| G1 | 2.91 g I-11 + 1.23 g S-3 | Ash violet |
| G1 | 2.91 g I-11 + 2.80 g S-4 | Blue-grey |
| G1 | 2.49 g I-15 + 1.09 g P-1 | Light violet-red |
| C1 | 2.49 g I-15 + 1.09 g P-2 | Light violet-red |
| G1 | 2.49 g I-15 + 1.38 g P-3 | Dark violet-red |
| C2 | 2.49 g I-15 + 1.09 g P-4 | Dark violet-brown |
| C1 | 2.49 g I-16 + 1.09 g P-1 | Uniform light golden brown |
| C1 | 2.49 g I-16 + 1.52 g P-2 | Uniform light brown |
| C1 | 2.49 g I-16 + 1.38 g P-3 | Uniform mid-brown |
| C1 | 2.49 g I-16 + 1.22 g P-4 | Uniform light brown |
| G1 | 2.91 g I-16 + 1.23 g P-5 | Light brown-orange |
| G1 | 2.49 g I-16 + 1.10 g S-1 | Light brown-red |
| C2 | 2.49 g I-16 + 1.09 g S-2 | Dark green |
| G1 | 2.49 g I-16 + 1.23 g S-3 | Blue-green |
| C2 | 2.49 g I-16 + 2.80 g S-4 | Dark blue violet |

In addition, the colors shown were obtained by combination of the oxidation dye precursors listed below:

| | | |
|---|---|---|
| I-3 + | P-2 | Ash violet |
| I-3 + | P-4 | Light brown-red |
| I-3 + | P-5 | Light blond-copper |
| I-4 + | P-1 | Light brown |
| I-4 + | P-2 | Dark grey |
| I-4 + | P-3 | Deep blue-black |
| I-4 + | P-4 | Dark grey-black |
| I-4 + | P-5 | Light ash brown |
| I-5 + | P-1 | Light brown-violet |
| I-5 + | P-2 | Ash grey-blue |
| I-5 + | P-3 | Deep blue-black |
| I-5 + | P-4 | Ash grey-blue |
| I-5 + | P-5 | Light golden blond |
| I-6 + | P-1 | Brown-red |
| I-6 + | P-2 | Dark violet |
| I-6 + | P-4 | Ash violet |
| I-6 + | P-5 | Ash red-violet |
| I-7 + | P-1 | Light golden blond |
| I-7 + | P-2 | Ash grey-brown |
| I-7 + | P-4 | Dark ash blond |
| I-7 + | P-5 | Light golden blond |
| I-8 + | P-1 | Light golden blond |
| I-8 + | P-2 | Light ash grey |
| I-8 + | P-3 | Dark blond |
| I-8 + | P-4 | Light ash blond |
| I-8 + | P-5 | Light blond |
| I-9 + | P-1 | Light blond-copper |
| I-9 + | P-2 | Medium ash grey |
| I-9 + | P-3 | Medium brown-violet |
| I-9 + | P-4 | Dark ash blond |
| I-9 + | P-5 | Light blond copper |
| I-12 + | S-1 | Silver grey-ashen |
| I-12 + | S-2 | Ash grey-ashen |
| I-12 + | S-3 | Green-grey |
| I-12 + | S-4 | Violet-grey |
| I-13 + | S-1 | Ashen |
| I-13 + | S-2 | Olive-violet |
| I-13 + | S-3 | Violet-grey |
| I-13 + | S-4 | Blue-violet |
| I-14 + | S-1 | Brown-violet |
| I-14 + | S-2 | Grey-green |
| I-14 + | S-3 | Violet-blue |
| I-14 + | S-4 | Blue-grey |
| I-15 + | S-1 | Light violet |
| I-15 + | S-2 | Light violet |
| I-15 + | S-3 | Light violet |
| I-15 + | S-4 | Dark violet |
| I-11 + | P-1 | Medium golden blond |
| I-11 + | P-2 | Dark blond |
| I-11 + | P-3 | Light brown |
| I-11 + | P-4 | Medium golden blond |
| I-11 + | P-5 | Light golden blond |
| I-12 + | P-1 | Medium golden blond |
| I-12 + | P-2 | Light ash blond |
| I-12 + | P-3 | Light brown |
| I-12 + | P-4 | Medium golden blond |
| I-12 + | P-5 | Light ash blond |
| I-13 + | P-1 | Medium golden blond |
| I-13 + | P-2 | Light ash violet |
| I-13 + | P-3 | Light brown |
| I-13 + | P-4 | Medium blond |
| I-13 + | P-5 | Light ash blond |
| I-14 + | P-1 | Medium golden blond |
| I-14 + | P-2 | Light ash blond |
| I-14 + | P-3 | Light brown |
| I-14 + | P-4 | Medium golden blond |
| I-14 + | P-5 | Light ash blond |
| I-10 + | P-1 | Light blond-copper |
| I-10 + | P-2 | Light golden blond |
| I-10 + | P-3 | Mid-brown |
| I-10 + | P-4 | Light golden blond |
| I-10 + | P-5 | Orange-red |

1. Oxidation colorants for coloring keratin fibers, characterized in that they contain as oxidation dye precursor at least one diaminoaniline corresponding to general formula (I): 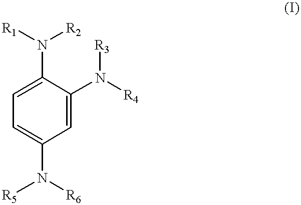
in which R₁ to R₆ independently of one another represent
hydrogen,
a (C₁₋₄)-alkyl group,
a hydroxy-(C₂₋₃)-alkyl group,
a (C₁₋₄)-alkoxy-(C₂₋₃)-alkyl group,
an amino-(C₂₋₃)-alkyl group, where the amino group may also bear one or two (C₁₋₄)-alkyl radicals, or
a 2,3-dihydroxypropyl group,
with the proviso that not all the substituents R₁ to R₆ simultaneously stand for hydrogen, and
R₁ and R₂ and/or R₃ and R₄ and/or R₅ and R₆ together with the nitrogen atom to which they are attached may also stand for an aziridine, acetidine, pyrrolidine, piperidine, azepan, azocine ring or a morpholino, thiomorpholino or piperazino group which, at the nitrogen atom, bears another substituent R₇ selected from hydrogen, a (C₁₋₄)-alkyl, a hydroxy-(C₂₋₃)-alkyl, a (C₁₋₄)-alkoxy-(C₂₋₃)-alkyl, an amino-(C₂₋₃)-alkyl or a 2,3-dihydroxypropyl group and the three remaining hydrogen atoms at the benzene ring independently of one another may even be replaced by a halogen atom or by a (C₁₋₄)-alkyl group,
or physiologically compatible salts thereof with inorganic and organic acids.

2. A colorant as claimed in claim 1, characterized in that at least two of the substituents R¹ to R⁶ are not hydrogen.

3. A formulation as claimed in claim 1 or 2, characterized in that at leat one of the groups —NR₁R₂, —NR₃R₄ or —NR₅R₆ stands for an aziridine, acetidine, pyrrolidine, piperidine, azepan, azocine ring or for a morpholino, thiomorpholino or piperazino group which, at the nitrogen atom, bears another substituent R₇ selected from hydrogen, a (C₁₋₄)-alkyl, a hydroxy-(C₂₋₃)-alkyl, a (C₁₋₄)-alkoxy-(C₂₋₃)-alkyl, an amino-(C₂₋₃)-alkyl or a 2,3-dihydroxypropyl group.

4. A colorant as claimed in any of claims 1 to 3, characterized in that the compound corresponding to formula (I) is present in quantities of 0.001 to 10% by weight and, more particularly, 0.1 to 5% by weight, based on the colorant as a whole.

5. A colorant as claimed in any of claims 1 to 4, characterized in that it additionally contains an oxidation dye precursor of the secondary intermediate type.

6. A colorant as claimed in any of claims 1 to 5, characterized in that it additionally contains an oxidation dye precursor of the primary intermediate type.

7. A colorant as claimed in any of claims 1 to 6, characterized in that ti additionally contains a substantive dye.

8. A colorant as claimed in any of claims 3 to 7, characterized in that it additionally contains a metal salt or a metal complex.

9. A colorant as claimed in claim 8, characterized in that the metal is selected from copper, manganese, cobalt, selenium, molybdenum, bismuth and ruthenium.

10. The use diaminoalkanes corresponding to general formula (I) in claim 1 for coloring keratin fibers.

25. The colorant of claim 24, further comprising a metal salt.

26. The colorant of cliam 24, further comprising a substantive dye.

27. A method of coloring keratin fiber, comprising coloring the fiber by contact of the keratin fiber with an effective amount of the colorant of claim 11.
